SEQUENCE LISTING (2 sequences)

SEQ ID NO: 1 - protein, 645 aa, Homo Sapiens
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 31 aa, Homo Sapiens
  VARIANT 31: Xaa = Any Amino Acid
HAEGTFTSDVSSYLEGQAAKEFIAWLVKGRX

(19) United States
(12) Patent Application Publication
(10) Pub. No.: US 2014/0378374 A1
(43) Pub. Date: Dec. 25, 2014

(54) METHODS OF TREATMENT

(71) Applicant: GlaxoSmithKline Intellectual Property (No.2) Limited, Middlesex (GB)

(72) Inventors: Murray Willis STEWART, Collegeville, PA (US); Yonghong F. Yang, King of Prussia, PA (US)

(21) Appl. No.: 14/310,471

(22) Filed: Jun. 20, 2014

Related U.S. Application Data
(60) Provisional application No. 61/952,301, filed on Mar. 13, 2014.
(60) Provisional application No. 61/837,978, filed on Jun. 21, 2013.

(51) Int. Cl. A61K 38/26

(52) U.S. Cl. CPC A61K 38/26
USPC 514/7.2

(57) ABSTRACT

The present invention provides methods of treating type 2 diabetes mellitus (T2DM) in human in need thereof comprising administering a pharmaceutical composition comprising a GLP-1 agonist to said human wherein said human has renal impairment. The present invention further provides methods of treating type 2 diabetes and/or providing glycemic control in a human wherein said human has renal impairment comprising administering to said human a subcutaneous injection of a pharmaceutical composition comprising albiglutide.

### CROSS REFERENCE TO RELATED APPLICATIONS

This application claims the benefit of U.S. Provisional Application No. 61/952,301, filed Mar. 13, 2014 and U.S. Provisional Application No. 61/837,978, filed Jun. 21, 2013.

### FIELD OF THE INVENTION

The present invention relates to pharmaceutical compositions and methods for administering GLP-1 agonists for the treatment of diabetes in patients with renal impairment.

### BACKGROUND

Chronic kidney disease (CKD) is a common comorbidity in patients with type 2 diabetes mellitus (T2DM) with an estimated prevalence of about 40% in this patient population. (USRDS 2013 Annual Data Report: Atlas of Chronic Kidney Disease and End-Stage Renal Disease in the United States. Bethesda, Md. 2013). Furthermore, diabetic nephropathy is the most common cause of end-stage renal disease, accounting for approximately 44% of all new cases in the United States. (USRDS 2013 Annual Data report, supra). Although intensive glycemic control can prevent or slow the progression of CKD in patients with T2DM (Patel A, et al. N Engl J Med. 2008; 358(24):2560-72.), pharmacokinetic changes due to reductions in GFR and kidney metabolism may require dose reductions for many antidiabetes medications or even contraindication of some agents in patients with CKD (Herrington, et al. QJM. 2013; 106(11):1059-61; Martinez-Castelao, et al. Nefrologia. 2012; 32(4):419-26; and Scheen, A J. Expert Opin Drug Metab Toxicol. 2013; 9(5):529-50). For example, metformin is associated with an increased risk of lactic acidosis and is contraindicated in patients with eGFR<60 ml/min/1.73 m² (although this threshold is controversial) (Herrington, et al. QJM. 2013; 106(11):1059-61; Martinez-Castelao, et al. Nefrologia. 2012; 32(4):419-26; and Scheen, A J. Expert Opin Drug Metab Toxicol. 2013; 9(5):529-50), and sulfonylureas should be used with caution because of an increased risk of hypoglycaemia (Scheen, A J. Expert Opin Drug Metab Toxicol. 2013; 9(5):529-50). Similar warnings and precautions about the use in patients with CKD are included in the labels of most other classes of anti-diabetes medications, including thiazolidinediones, dipeptidyl peptidase 4 (DPP-4) inhibitors (except for linagliptin), sodium-glucose co-transporter 2 inhibitors, glucagon-like peptide 1 receptor (GLP-1R) agonists, and basal insulin (Martinez-Castelao, et al. Nefrologia. 2012; 32(4):419-26).

Hypoglycemic agents may be used in the treatment of both type 1 and type 2 diabetes to lower glucose concentration in blood. Insulinotropic peptides have been implicated as possible therapeutic agents for the treatment of diabetes. Insulinotropic peptides include, but are not limited to, incretin hormones, for example, gastric inhibitory peptide (GIP) and glucagon like peptide-1 (GLP-1), as well as fragments, variants, and/or conjugates thereof. Insulinotropic peptides also include, for example, exendin 3 and exendin 4. GLP-1 is a 36 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of β-cells. In non-clinical experiments GLP-1 promotes continued beta cell competence by stimulating transcription of genes important for glucose dependent insulin secretion and by promoting beta-cell neogenesis (Meier, et al. Biodrugs. 2003; 17 (2): 93-102).

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption.

In people with type 2 diabetes mellitus (T2DM), the normal post-prandial rise in GLP-1 is absent or reduced (Vilsboll T, et al., Diabetes. 2001. 50; 609-613). Accordingly, one rationale for administering exogenous GLP-1, an incretin hormone, or an incretin mimetic, is to enhance, replace or supplement endogenous GLP-1 in order to increase meal-related insulin secretion, reduce glucagon secretion, and/or slow gastrointestinal motility. Native GLP-1 has a very short serum half-life (<5 minutes). Accordingly, it is not currently feasible to exogenously administer native GLP-1 as a therapeutic treatment for diabetes. Commercially available incretin mimetics such as Exenatide (Byetta®) improve glycemic control by reducing fasting and postprandial glucose concentrations when administered subcutaneously (5 μg or 10 μg BID) to patients with T2DM.

Current therapies available for patients with type 2 diabetes and renal impairment are limited. Several medications are contraindicated in these patients and many require dose adjustment. Albiglutide is a novel, long-acting GLP-1 receptor agonist composed of a DPP-4-resistant GLP-1 dimer fused to recombinant human albumin. These modifications confer an extended plasma half-life and allow once weekly dosing (Bush, et al. Diabetes, obesity & metabolism. 2009; 11(5):498-505 and Matthews, et al. J Clin Endocrinol Metab. 2008; 93(12):4810-7.). Results from Phase 3 clinical trials in patients with T2DM demonstrated that weekly dosing with albiglutide as monotherapy or in combination with other antidiabetes medication was effective and well tolerated (Leiter, et al. American Diabetes Association 73rd Scientific Sessions; 2013; Chicago, Ill.; Pratley, et al. American Diabetes Association 73rd Scientific Sessions; 2013; Chicago, Ill.; Home et al. American Diabetes Association 73rd Scientific Sessions; 2013; Chicago, Ill. And Nauck, et al. American Diabetes Association 73rd Scientific Sessions; 2013; Chicago, Ill.). Albiglutide has an extended half-life of approximately 5 days, which allows for once-weekly (QW) dosing. Albiglutide has been shown to have lower gastrointestinal adverse events when administered to humans compared with other GLP-1 agonists such as exendin-4 (see, for instance, WO2010/068735).

Thus, there is an unmet need for methods of treating type 2 diabetes and/or providing glycemic control in patients with renal impairment.

### BRIEF DESCRIPTION OF THE DRAWINGS

FIG. 1. SEQ ID NO.:1.

FIG. 2: Primary Endpoint: A1C Over Time Through Week 26 (ITT-LOCF).

FIG. 3: Model-adjusted Change from Baseline in A1C at Week 26 by severity of Renal Impairment.

FIG. 4: FPG Over Time Through Week 26 (ITT-LOCF).

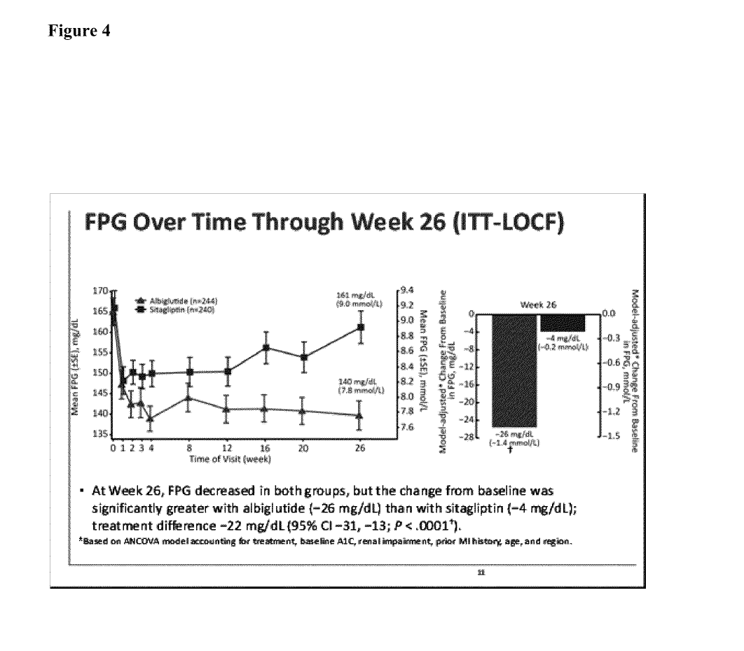

FIG. 5: Weight Over Time through Week 26 (ITT-LOCF).

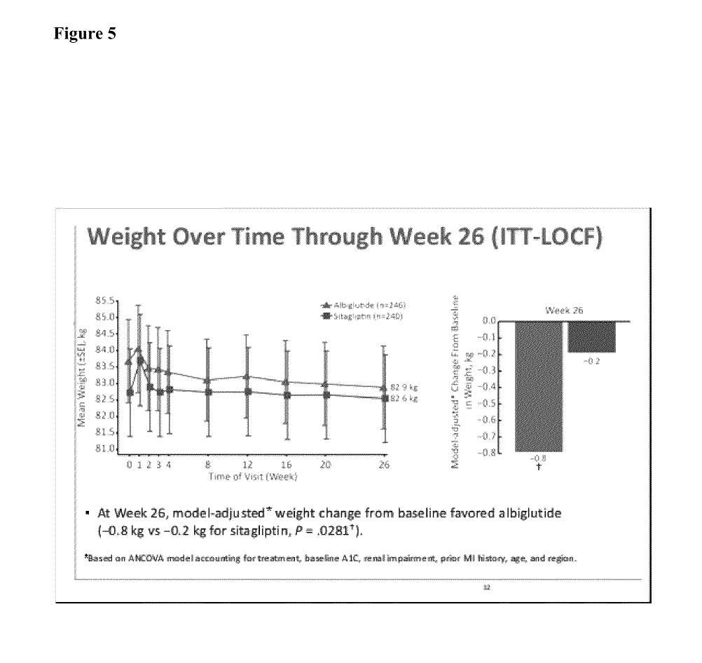

FIG. 6: Clinically Meaningful A1C Response at 26 Weeks (ITT-LOCF)

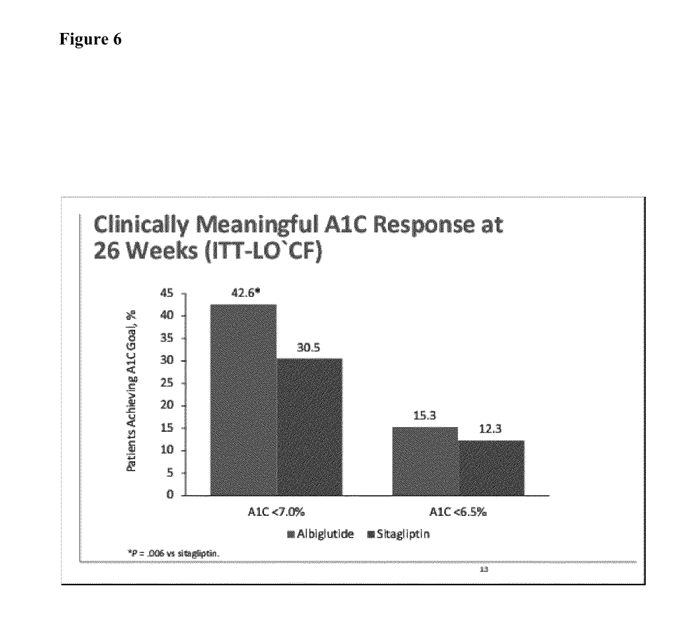

FIG. 7. Representative Plot of Individual Plasma Concentrations of Albiglutide and Albumin Versus Time in a Participant (Single Dose Study) Requiring Repeated Hemodialysis.

FIG. 8. Albiglutide Clearance vs. EGFR mL/min

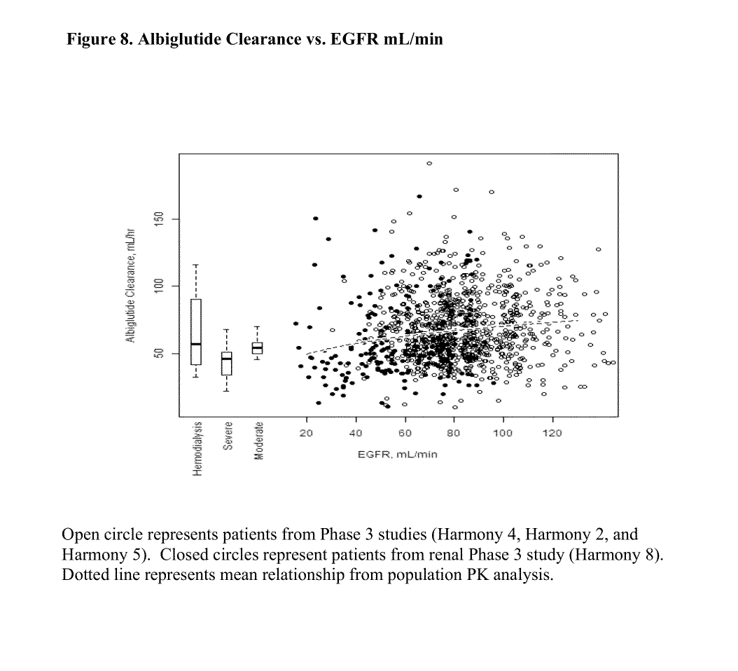

FIG. 9: HbA1c and FPG Changes from Baseline as a Function of Renal Impairment in the Phase 3 Pooled Analysis

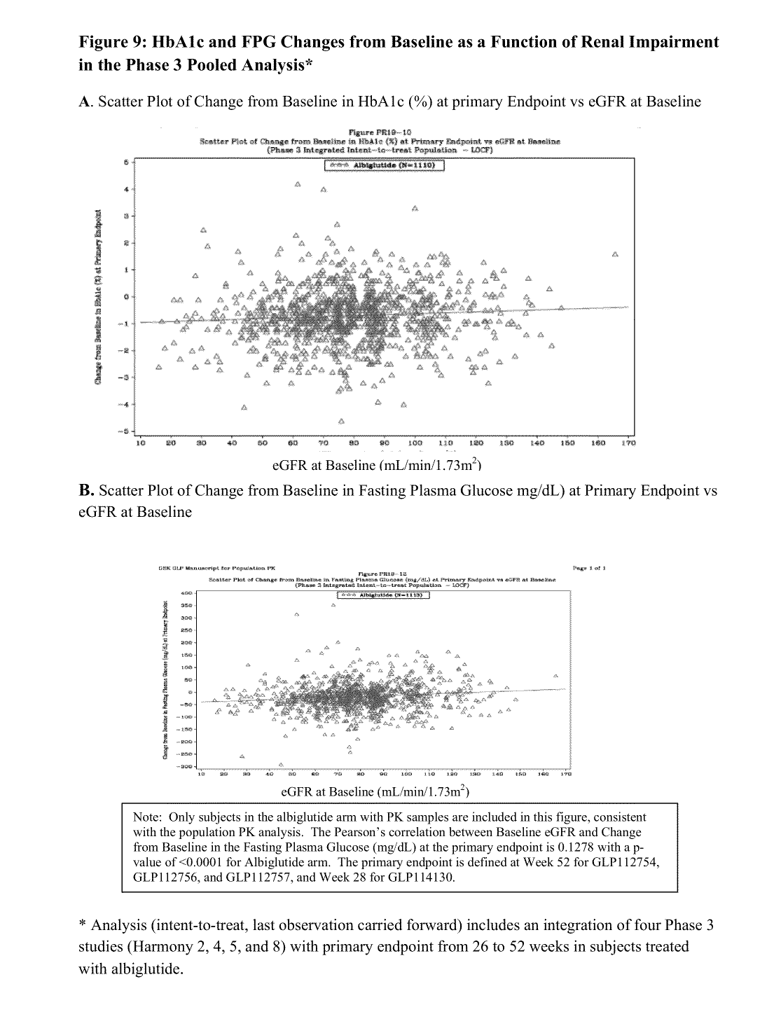

### SUMMARY OF THE INVENTION

The present invention provides methods of treating type 2 diabetes in human in need thereof comprising administering a pharmaceutical composition comprising a GLP-1 agonist to said human wherein said human has renal impairment.

The present invention further provides methods of treating type 2 diabetes in a human wherein said human has renal impairment comprising administering to said human a subcutaneous injection of a pharmaceutical composition comprising albiglutide. In particular, the present invention comprises providing glycemic control in a human with renal impairment with type 2 diabetes mellitus comprising administering to said human a composition comprising albiglutide.

### DEFINITIONS

As used herein “GLP-1 agonist” and “GLP-1R agonist” and “GLP-1 receptor agonist” and grammatical variations thereof are used synonymously to mean any compound, agent, polypeptide and/or incretin mimetic that has at least one GLP-1 activity. Examples of GLP-1 agonists include, but are not limited to, albiglutide, and variants thereof, liraglutide, Byetta, Bydureon, exenatide, exendin 3 and exendin 4.

“GLP-1 agonist composition” as used herein means any composition capable of stimulating the secretion of insulin, or otherwise raising the level of insulin, including, but not limited to an incretin hormone and an incretin mimetic.

“Incretin hormone” as used herein means any hormone that potentiates insulin secretion or otherwise raises the level of insulin in a mammal. One example of an incretin hormone is GLP-1. GLP-1 is an incretin secreted by intestinal L cells in response to ingestion of food. In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying time and slows small bowel motility delaying food absorption. GLP-1 promotes continued beta cell competence by stimulating transcription of genes involved in glucose dependent insulin secretion and by promoting beta-cell neogenesis (Meier, et al. Biodrugs 2003; 17 (2): 93-102).

“GLP-1 activity” as used herein means one or more of the activities of naturally occurring human GLP-1, including but not limited to, reducing blood and/or plasma glucose, stimulating glucose-dependent insulin secretion or otherwise raising the level or insulin, suppressing glucagon secretion, reducing fructosamine, increases glucose delivery and metabolism to the brain, delaying gastric emptying, and promoting beta cell competence, and/or neogenesis. Any of these activities and other activity associated with GLP-1 activity may be caused directly or indirectly by a composition having GLP-1 activity or a GLP-1 agonist. By way of example, a composition having GLP-1 activity may directly or indirectly stimulate glucose-dependent while the stimulation of insulin production may indirectly reduce plasma glucose levels in a mammal.

An “incretin mimetic” as used herein is a compound capable of potentiating insulin secretion or otherwise raise the level or insulin. An incretin mimetic may be capable of stimulating insulin secretion, increasing beta cell neogenesis, inhibiting beta cell apoptosis, inhibiting glucagon secretion, delaying gastric emptying and inducing satiety in a mammal. An incretin mimetic may include, but is not limited to, any polypeptide which has GLP-1 activity, including but not limited to, exendin 3 and exendin 4, including any fragments and/or variants and/or conjugates thereof.

“Hypoglycemic agent” as used herein means any compound or composition comprising a compound capable of reducing blood glucose. A hypoglycemic agent may include, but is not limited to, any GLP-1 agonist including incretin hormones or incretin mimetics, GLP-1 and/or fragment, variant and/or conjugate thereof. Other hypoglycemic agents include, but are not limited to, drugs that increase insulin secretion (e.g., sulfonylureas (SU) and meglitinides), inhibit GLP-1 break down (e.g., DPP-IV inhibitors, such as sitagliptin), increase glucose utilization (e.g., glitazones, thiazolidinediones (TZDs) and/or pPAR agonists), reduce hepatic glucose production (e.g., metformin), and delay glucose absorption (e.g., α-glucosidase inhibitors). Examples of sulfonylureas include but are not limited to acetohexamide, chlorpropamide, tolazamide, glipizide, gliclazide, glibenclamide (glyburide), gliquidone, and glimepiride. Examples of glitazones include, but are not limited to, rosiglitazone and pioglitazone.

“Polynucleotide(s)” generally refers to any polyribonucleotide or polydeoxyribonucleotide that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques that are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth. Enzymol. (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants may also include, but are not limited to, polypeptides or fragments thereof having chemical modification of one or more of its amino acid side groups. A chemical modification includes, but is not limited to, adding chemical moieties, creating new bonds, and removing chemical moieties. Modifications at amino acid side groups include, without limitation, acylation of lysine-ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist.

As used herein “albiglutide,” “ALB,” and “Albi” refers to a recombinant fusion protein consisting of 2 copies of a 30-amino acid sequence of modified human glucagon-like peptide 1 (GLP-1, fragment 7-36(A8G)) genetically fused in series to recombinant human serum albumin. The amino acid sequence of albiglutide is shown below as SEQ ID NO:1.

(SEQ ID NO: 1)

| | |
|---|---|
| HGEGTFTSDVSSYLEGQAAKEFIAWLVKGRHGEGTFTSDVSSYLEG | 60 |
| QAAKEFIAWLVKGR | |
| DAHKSEVAHRFKDLGEENFKALVLIAFAQYLQQCPFEDHVKLVNEV | 120 |
| TEFAKTCVADESAE | |
| NCDKSLHTL FGDKLCTVATLRETYGEMADCCAKQEPERNECFLQH | 180 |
| KDDNPNLPRLVRPE | |
| VDVMCTAFHDNEETFLKKYLYEIARRHPYFYAPELLFFAKRYKAAF | 240 |
| TECCQAADKAACLL | |
| PKLDELRDEGKASSAKQRLKCASLQKFGERAFKAWAVARLSQRFPK | 300 |
| AEFAEVSKLVTDLT | |
| KVHTECCHGDLLECADDRADLAKYICENQDSISSKLKECCEKPLLE | 360 |
| KSHCIAEVENDEMP | |
| ADLPSLAADFVESKDVCKNYAEAKDVFLGMFLYEYARRHPDYSVVL | 420 |
| LLRLAKTYETTLEK | |
| CCAAADPHECYAKVFDEFKPLVEEPQNLIKQNCELFEQLGEYKFQN | 480 |
| ALLVRYTKKVPQVS | |
| TPTLVEVSRNLGKVGSKCCKHPEAKRMPCAEDYLSVVLNQLCVLHE | 540 |
| KTPVSDRVTKCCTE | |
| SLVNRRPCFSALEVDETYVPKEFNAETFTFHADICTLSEKERQIKK | 600 |
| QTALVELVKHKPKA | |
| TKEQLKAVMDDFAAFVEKCCKADDKETCFAEEGKKLVAASQAALGL | 674 |

As used herein “truncated” polypeptide refers to a first polypeptide having substantially the same amino acid sequence over the entire length of a second polypeptide, but wherein the first polypeptide has either a shortened N-terminus and/or C-terminus compared with the second polypeptide. By way of example, the first polypeptide may be missing amino acids 1, 2, 3, 4, 5, 6, 7, 8, 9, or 10 from its sequence compared with the second polypeptide; however, the amino acid sequence for both polypeptides may be identical once aligned, excluding the truncated section. Similarly, a first polypeptide could have a truncated amino acid sequence when compared with a second polypeptide at its C-terminus. For instance, a polypeptide having the amino acid sequence set forth in SEQ ID NO:1 which is truncated at the N-terminus by 5 amino acids would consist of amino acids 6 to 645 of SEQ ID NO:1. Whereas a polypeptide having the amino acid sequence set forth in SEQ ID NO:1 which is truncated at the C-terminus by 5 amino acids will consist of amino acids 1 to 640 of SEQ ID NO:1.

As used herein “fragment,” when used in reference to a polypeptide, is a polypeptide having an amino acid sequence that is the same as part but not all of the amino acid sequence of the entire naturally occurring polypeptide. Fragments may be “free-standing” or comprised within a larger polypeptide of which they form a part or region as a single continuous region in a single larger polypeptide. By way of example, a fragment of naturally occurring GLP-1 would include amino acids 7 to 36 of naturally occurring amino acids 1 to 36. Furthermore, fragments of a polypeptide may also be variants of the naturally occurring partial sequence. For instance, a fragment of GLP-1 comprising amino acids 7-30 of naturally occurring GLP-1 may also be a variant having amino acid substitutions within its partial sequence.

As used herein “conjugate” or “conjugated” refers to two molecules that are bound to each other. For example, a first polypeptide may be covalently or non-covalently bound to a second polypeptide. The first polypeptide may be covalently bound by a chemical linker or may be genetically fused to the second polypeptide, wherein the first and second polypeptide share a common polypeptide backbone.

As used herein “tandemly oriented” refers to two or more polypeptides that are adjacent to one another as part of the same molecule. They may be linked either covalently or non-covalently. Two or more tandemly oriented polypeptides may form part of the same polypeptide backbone. Tandemly oriented polypeptides may have direct or inverted orientation and/or may be separated by other amino acid sequences.

As used herein, “reduce” or “reducing” blood or plasma glucose refers to a decrease in the amount of blood glucose observed in the blood of a patient after administration a hypoglycemic agent. Reductions in blood or plasma glucose can be measured and assessed per individual or as a mean change for a group of subjects. Additionally, mean reductions in blood or plasma glucose can be measured and assessed for a group of treated subjects as a mean change from baseline and/or as a mean change compared with the mean change in blood or plasma glucose among subjects administered placebo.

As used herein “enhancing GLP-1 activity” refers to an increase in any and all of the activities associated with naturally occurring GLP-1. By way of example, enhancing GLP-1 activity can be measured after administration of at least one polypeptide having GLP-1 activity to a subject and compared with GLP-1 activity in the same subject prior to the administration of the polypeptide having GLP-1 activity or in comparison to a second subject who is administered placebo.

As used herein “diseases associated with elevated blood glucose” include, but are not limited to, type 1 and type 2 diabetes, glucose intolerance, and hyperglycemia.

As used herein “co-administration” or “co-administering” refers to administration of two or more compounds or two or more doses of the same compound to the same patient. Co-administration of such compounds may be simultaneous or at about the same time (e.g., within the same hour) or it may be within several hours or days of one another. For example, a first compound may be administered once weekly while a second compound is co-administered daily.

As used herein “maximum plasma concentration” or “Cmax” means the highest observed concentration of a substance (for example, a polypeptide having GLP-1 activity or a GLP-1 agonist) in mammalian plasma after administration of the substance to the mammal.

As used herein “Area Under the Curve” or “AUC” is the area under the curve in a plot of the concentration of a substance in plasma against time. AUC can be a measure of the integral of the instantaneous concentrations during a time interval and has the units mass×time/volume, which can also be expressed as molar concentration×time such as nM×day. AUC is typically calculated by the trapezoidal method (e.g., linear, linear-log). AUC is usually given for the time interval zero to infinity, and other time intervals are indicated (for example AUC (t1,t2) where t1 and t2 are the starting and finishing times for the interval). Thus, as used herein “AUC₀₋₂₄ₕ” refers to an AUC over a 24-hour period, and “AUC₀₋₄ₕ” refers to an AUC over a 4-hour period.

As used herein “weighted mean AUC” is the AUC divided by the time interval over which the time AUC is calculated. For instance, weighted mean AUC₀₋₂₄ₕ would represent the AUC₀₋₂₄ₕ divided by 24 hours.

As used herein “confidence interval” or “CI” is an interval in which a measurement or trial falls corresponding to a given probability p where p refers to a 90% or 95% CI and are calculated around either an arithmetic mean, a geometric mean, or a least squares mean. As used herein, a geometric mean is the mean of the natural log-transformed values back-transformed through exponentiation, and the least squares mean may or may not be a geometric mean as well but is derived from the analysis of variance (ANOVA) model using fixed effects.

As used herein the “coefficient of variation (CV)” is a measure of dispersion and it is defined as the ratio of the standard deviation to the mean. It is reported as a percentage (%) by multiplying the above calculation by 100 (% CV).

As used herein “Tmax” refers to the observed time for reaching the maximum concentration of a substance in plasma of a mammal after administration of that substance to the mammal.

As used herein “serum or plasma half life” refers to the time required for half the quantity of a substance administered to a mammal to be metabolized or eliminated from the serum or plasma of the mammal by normal biological processes.

As used herein “dose” refers to any amount of therapeutic compound which may be administered to a mammal, including a human. An effective dose is a dose of a compound that is in an amount sufficient to induce at least one of the intended effects of the therapeutic compound. For instance, an effective dose of a GLP-1 agonist would induce at least one type of GLP-1 activity in a human when administered to a human, such as, but not limited to providing glycemic control and increasing insulin production in said human. As is understood in the art, an effective dose of a therapeutic compound can be measured by a surrogate endpoint. Thus, by way of another example, an effective dose of a GLP-1 agonist can be measured by its ability to lower serum glucose in a human.

As used herein “renal impairment” refers to a condition in which a patient's kidneys are unable to adequately filter waste products from the blood. Renal impairment can either be chronic or acute. Certain disease including diabetes, high blood pressure, heart failure, obesity and long-term kidney disease can increase the chance of a patient developing renal impairment. Renal impairment can be measured by several criteria understood by those skilled in the art. One methods for determining renal impairment is to measure estimated Glomerular Filtration Rate (eGFR). The glomerular filtration rate measures how well a patient's kidneys filter the wastes from blood. The higher the filtration rate, the better the kidneys are working. Normal filtration rate is about 90-100 millilitres per minute, or 100 mL/min. eGFR over 60 mL/min/1.73 m², indicates kidney function which is normal or close to normal. Patients with eGFR above 60 mL/min/1.73 m², may be diagnosed with chronic kidney disease (CKD). A value below 60 mL/min/1.73 m², suggests some loss of kidney function. eGFR can be measures by several methods known in the art, including Modification of Diet in Renal disease (MDRD), CKD-EPI formula and the Mayo Quadratic formula. As is also understood in the art creatinine clearance rate can also be used to approximate GFR and renal clearance.

As used herein “statistically significant” refers a less than 0.05 p-value for the statistical comparison of treatment difference between one treatment vs. another treatment in randomized controlled clinical trial.

Sitagliptin is marketed as the phosphate salt under the trade name Januvia® by Merck & Co. It is an oral antihyperglycemic (hypoglycaemic agent or antidiabetic drug) of the dipeptidyl peptidase-4 (DPP-4) inhibitor class. This enzyme-inhibiting drug is used either alone or in combination with other oral antihyperglycemic agents (such as metformin or a thiazolidinedione) for treatment of diabetes mellitus type 2. The chemical structure of sitagliptin is presented in Formula I below:

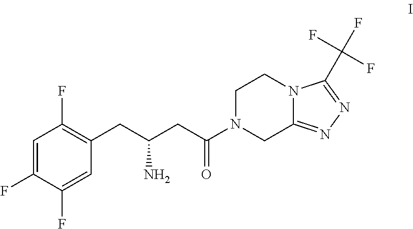 [SMILES: N[C@@H](CC(=O)N1CCn2c(nnc2C(F)(F)F)C1)Cc1cc(F)c(F)cc1F]

Liraglutide, marketed under the brand name Victoza, is a long-acting glucagon-like peptide-1 agonist (GLP-1 agonist) developed by Novo Nordisk for the treatment of type 2 diabetes. Liraglutide is an acylated human glucagon-like peptide-1 (GLP-1) agonist, with a 97% amino acid sequence identity to endogenous human GLP-1(7-37). GLP-1(7-37) represents less than 20% of total circulating endogenous GLP-1. Like GLP-1(7-37), liraglutide activates the GLP-1 receptor, a membrane-bound cell-surface receptor coupled to adenylyl cyclase by the stimulatory G-protein, Gs, in pancreatic beta cells. Liraglutide increases intracellular cyclic AMP (cAMP), leading to insulin release in the presence of elevated glucose concentrations.²⁵

Exenatide (marketed as Byetta or Bydureon) is a glucagon-like peptide-1 agonist (GLP-1 agonist) medication, belonging to the group of incretin mimetics, approved in April 2005 for the treatment of diabetes mellitus type 2. Exenatide in its Byetta form is administered as a subcutaneous injection (under the skin) of the abdomen, thigh, or arm, any time within the 60 minutes before the first and last meal of the day.²⁶ A once-weekly injection has been approved as of Jan. 27, 2012 under the trademark Bydureon. It is manufactured by Amylin Pharmaceuticals and commercialized by Astrazeneca.

### DETAILED DESCRIPTION OF THE INVENTION

The present invention provides methods of treating type 2 diabetes mellitus in a human in need thereof comprising administering a pharmaceutical composition comprising a GLP-1 agonist to said human wherein said human has renal impairment. In one embodiment, treating T2DM means providing glycemic control for the human in need thereof. In one embodiment, the GLP-1 agonist comprises a polypeptide having at least 95% sequence identity to the amino acid sequence set forth SEQ ID NO:1 over the entire sequence of SEQ ID NO:1. In one embodiment, the GLP-1 agonist comprises a polypeptide having at least 99% sequence identity to the amino acid sequence set forth SEQ ID NO:1 over the entire sequence of SEQ ID NO:1. In one embodiment, the GLP-1 agonist consists of a polypeptide having the amino acid sequence set forth in SEQ ID NO:1. In one embodiment, the GLP-1 agonist consists of a polypeptide having the amino acid sequence set forth in SEQ ID NO:1 which is truncated at the N-terminus and/or the C-terminus by 1, 2, 3, 4, or 5 amino acids.

An embodiment of the invention comprises a polypeptide that may be, but is not limited to, GLP-1 or a fragment, variant, and/or conjugate thereof. GLP-1 fragments and/or variants and/or conjugates of the present invention typically have at least one GLP-1 activity. A GLP-1 or a fragment, variant, and/or conjugate thereof may comprise human serum albumin. Human serum albumin may be conjugated to the GLP-1 or fragment and/or variant thereof. Human serum albumin may be conjugated to an incretin hormone (such as GLP-1) and/or incretin mimetic (such as exendin 3 and exendin 4) and/or fragments and/or variants thereof through a chemical linker prior to injection or may be chemically linked to naturally occurring human serum albumin in vivo (see for instance, U.S. Pat. No. 6,593,295 and U.S. Pat. No. 6,329,336, herein incorporated by reference in their entirety). Alternatively, human serum albumin may be genetically fused to a GLP-1 and/or fragment and/or variant thereof or other GLP-1 agonist such as exendin-3 or exendin-4 and/or fragments and/or variants thereof. Examples of GLP-1 and fragments and/or variants thereof fused with human serum albumin are provided in the following: WO 2003/060071, WO 2003/59934, WO 2005/003296, WO 2005/077042 and U.S. Pat. No. 7,141,547 (herein incorporated by reference in their entirety).

Polypeptides having GLP-1 activity may comprise at least one fragment and/or variant of human GLP-1. The two naturally occurring fragments of human GLP-1 are represented in SEQ ID NO: 2.

(SEQ ID NO.: 2)
7 8 9 10 11 12 13 14 15 16 17
His-Ala-Glu-Gly-Thr-Phe-Thr-Ser-Asp-Val-Ser-
18 19 20 21 22 23 24 25 26 27 28
Ser-Tyr-Leu-Glu-Gly-Gln-Ala-Ala-Lys-Glu-Phe-
29 30 31 32 33 34 35 36 37
Ile-Ala-Trp-Leu-Val-Lys-Gly-Arg-Xaa

wherein: Xaa at position 37 is Gly (hereinafter designated as “GLP-1(7-37)”), or —NH₂ (hereinafter designated as “GLP-1(7-36)”). GLP-1 fragments may include, but are not limited to, molecules of GLP-1 comprising, or alternatively consisting of, amino acids 7 to 36 of human GLP-1 (GLP-1(7-36)). Variants of GLP-1 or fragments thereof may include, but are not limited to, one, two, three, four, five or more amino acid substitutions in wild type GLP-1 or in the naturally occurring fragments of GLP-1 shown in SEQ ID NO.: 2. Variants GLP-1 or fragments of GLP-1 may include, but are not limited to, substitutions of an alanine residue analogous to alanine 8 of wild type GLP-1, such alanine being mutated to a glycine (hereinafter designated as “A8G”) (See for example, the mutants disclosed in U.S. Pat. No. 5,545,618, herein incorporated by reference in its entirety).

In some aspects, at least one fragment and variant of GLP-1 comprises GLP-1(7-36(A8G)) and is genetically fused to human serum albumin. In a further embodiment, polypeptides of the invention comprise one, two, three, four, five, or more tandemly oriented molecules of GLP-1 and/or fragments and/or variants thereof fused to the N- or C-terminus of human serum albumin or variant thereof. Other embodiments have such A8G polypeptides fused to the N- or C-terminus of albumin or variant thereof. An example of two tandemly oriented GLP-1(7-36)(A8G) fragments and/or variants fused to the N-terminus of human serum albumin comprises SEQ ID NO:1, which is presented in

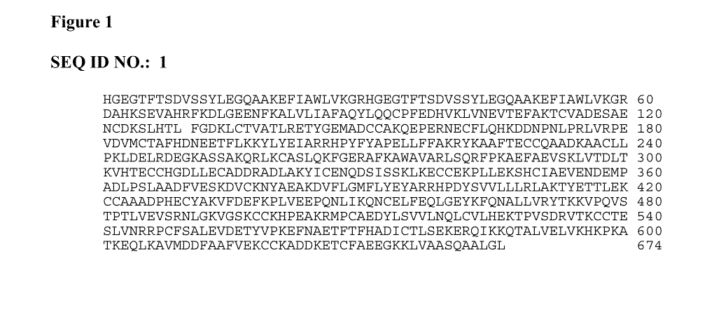

FIG. 1. In another aspect, at least one fragment and variant of GLP-1 comprises at least two GLP-1(7-36(A8G)) tandemly and genetically fused to the human serum albumin. At least two GLP-1(7-36(A8G)) may be genetically fused at the N-terminus of the human serum albumin. At least one polypeptide having GLP-1 activity may comprise SEQ ID No.: 1.

Variants of GLP-1(7-37) may be denoted for example as Glu²²-GLP-1(7-37)OH which designates a GLP-1 variant in which the glycine normally found at position 22 of GLP-1(7-37)OH has been replaced with glutamic acid; Val⁸-Glu²²-GLP-1(7-37)OH designates a GLP-1 compound in which alanine normally found at position 8 and glycine normally found at position 22 of GLP-1(7-37)OH have been replaced with valine and glutamic acid, respectively. Examples of variants of GLP-1 include, but are not limited to,

| | | |
|---|---|---|
| Val⁸-GLP-1(7-37)OH | Gly⁸-GLP-1(7-37)OH | Glu²²-GLP-1(7-37)O—H |
| Asp²²-GLP-1(7-37)OH | Arg²²-GLP-1(7-37)OH | Lys²²-GLP-1(7-37)OH |
| Cys²²-GLP-1(7-37)OH | Val⁸-Glu²²-GLP-1(7-37)OH | Val⁸-Asp²²-GLP-1(7-37)OH |
| Val⁸-Arg²²-GLP-1(7-37)OH | Val⁸-Lys²²-GLP-1(7-37)OH | Val⁸-Cys²²-GLP-1(7-37)OH |
| Gly⁸-Glu²²-GLP-1(7-37)OH | Gly⁸-Asp²²-GLP-1(7-37)OH | Gly⁸-Arg²²-GLP-1(7-37)OH |
| Gly⁸-Lys²²-GLP-1(7-37)OH | Gly⁸-Cys²²-GLP-1(7-37)OH | Glu²²-GLP-1(7-36)OH |
| Asp²²-GLP-1(7-36)OH | Arg²²-GLP-1(7-36)OH | Lys²²-GLP-1(7-36)OH |
| Cys²²-GLP-1(7-36)OH | Val⁸-Glu²²-GLP-1(7-36)OH | Val⁸-Asp²²-GLP-1(7-36)OH |
| Val⁸-Arg²²-GLP-1(7-36)OH | Val⁸-Lys²²-GLP-1(7-36)OH | Val⁸-Cys²²-GLP-1(7-36)OH |
| Gly⁸-Glu²²-GLP-1(7-36)OH | Gly⁸-Asp²²-GLP-1(7-36)OH | Gly⁸-Arg²²-GLP-1(7-36)OH |
| Gly⁸-Lys²²-GLP-1(7-36)OH | Gly⁸-Cys²²-GLP-1(7-36)OH | Lys²³-GLP-1(7-37)OH |
| Val⁸-Lys²³-GLP-1(7-37)OH | Gly⁸-Lys²³-GLP-1(7-37)OH | His²⁴-GLP-1(7-37)OH |
| Val⁸-His²⁴-GLP-1(7-37)OH | Gly⁸-His²⁴-GLP-1(7-37)OH | Lys²⁴-GLP-1(7-37)OH |
| Val⁸-Lys²⁴-GLP-1(7-37)OH | Gly⁸-Lys²³-GLP-1(7-37)OH | Glu³⁰-GLP-1(7-37)OH |
| Val⁸-Glu³⁰-GLP-1(7-37)OH | Gly⁸-Glu³⁰-GLP-1(7-37)OH | Asp³⁰-GLP-1(7-37)OH |
| Val⁸-Asp³⁰-GLP-1(7-37)OH | Gly⁸-Asp³⁰-GLP-1(7-37)OH | Gln³⁰-GLP-1(7-37)OH |
| Val⁸-Gln³⁰-GLP-1(7-37)OH | Gly⁸-Gln³⁰-GLP-1(7-37)OH | Tyr³⁰-GLP-1(7-37)OH |
| Val⁸-Tyr³⁰-GLP-1(7-37)OH | Gly⁸-Tyr³⁰-GLP-1(7-37)OH | Ser³⁰-GLP-1(7-37)OH |
| Val⁸-Ser³⁰-GLP-1(7-37)OH | Gly⁸-Ser³⁰-GLP-1(7-37)OH | His³⁰-GLP-1(7-37)OH |
| Val⁸-His³⁰-GLP-1(7-37)OH | Gly⁸-His³⁰-GLP-1(7-37)OH | Glu³⁴-GLP-1(7-37)OH |
| Val⁸-Glu³⁴-GLP-1(7-37)OH | Gly⁸-Glu³⁴-GLP-1(7-37)OH | Ala³⁴-GLP-1(7-37)OH |
| Val⁸-Ala³⁴-GLP-1(7-37)OH | Gly⁸-Ala³⁴-GLP-1(7-37)OH | Gly³⁴-GLP-1(7-37)OH |
| Val⁸-Gly³⁴-GLP-1(7-37)OH | Gly⁸-Gly³⁴-GLP-1(7-37)OH | Ala³⁵-GLP-1(7-37)OH |
| Val⁸-Ala³⁵-GLP-1(7-37)OH | Gly⁸-Ala³⁵-GLP-1(7-37)OH | Lys³⁵-GLP-1(7-37)OH |
| Val⁸-Lys³⁵-GLP-1(7-37)OH | Gly⁸-Lys³⁵-GLP-1(7-37)OH | His³⁵-GLP-1(7-37)OH |
| Val⁸-His³⁵-GLP-1(7-37)OH | Gly⁸-His³⁵-GLP-1(7-37)OH | Pro³⁵-GLP-1(7-37)OH |
| Val⁸-Pro³⁵-GLP-1(7-37)OH | Gly⁸-Pro³⁵-GLP-1(7-37)OH | Glu³⁵-GLP-1(7-37)OH |
| Gly⁸-Glu³⁵-GLP-1(7-37)OH | Val⁸-Ala²⁷-GLP-1(7-37)OH | Val⁸-His³⁷-GLP-1(7-37)OH |
| Val⁸-Glu²²-Lys²³-GLP-1(7-37)OH | Val⁸-Glu²²-Glu²³-GLP-1(7-37)OH | Val⁸-Glu²²-Ala²⁷-GLP-1(7-37)OH |
| Val⁸-Gly³⁴-Lys³⁵-GLP-1(7-37)OH | Val⁸-His³⁷-GLP-1-(7-37)OH | Gly⁸-His³⁷-GLP-1(7-37)OH |
| Val⁸-Glu²²-Ala²⁷-GLP-1(7-37)OH | Gly⁸-Glu²²-Ala²⁷-GLP-1(7-37)OH | Val⁸-Lys²²-Glu²³-GLP-1(7-37)OH |
| Gly⁸-Lys²²-Glu²³-GLP-1(7-37)OH. | Val⁸-Glu³⁵-GLP-1(7-37)OH | |

Variants of GLP-1 may also include, but are not limited to, GLP-1 or GLP-1 fragments having chemical modification of one or more of its amino acid side groups. A chemical modification includes, but is not limited to, adding chemical moieties, creating new bonds, and removing chemical moieties. Modifications at amino acid side groups include, without limitation, acylation of lysine-ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist.

GLP-1 fragments or variants may also include polypeptides in which one or more amino acids have been added to the N-terminus and/or C-terminus of GLP-1(7-37)OH of said fragment or variant. The amino acids in GLP-1 in which amino acids have been added to the N-terminus or C-terminus are denoted by the same number as the corresponding amino acid in GLP-1(7-37)OH. For example, the N-terminus amino acid of a GLP-1 compound obtained by adding two amino acids to the N-terminus of GLP-1(7-37)OH is at position 5; and the C-terminus amino acid of a GLP-1 compound obtained by adding one amino acid to the C-terminus of GLP-1(7-37)OH is at position 38. Thus, position 12 is occupied by phenylalanine and position 22 is occupied by glycine in both of these GLP-1 compounds, as in GLP-1(7-37)OH. Amino acids 1-6 of a GLP-1 with amino acids added to the N-terminus may be the same as or a conservative substitution of the amino acid at the corresponding position of GLP-1(1-37)OH. Amino acids 38-45 of a GLP-1 with amino acids added to the C-terminus may be the same as or a conservative substitution of the amino acid at the corresponding position of glucagon or exendin-4.

In another embodiment, the pharmaceutical composition of the present invention can administered to a human once daily, once every other day, once every seven days, once every fourteen days, once every four weeks, and/or once every month. In another aspect, the pharmaceutical compositions comprises at least 30 mg/mL of SEQ ID NO:1. In another aspect, the pharmaceutical composition consists of 30 mg/mL of SEQ ID NO:1, sodium phosphate, trehalose, mannitol, TWEEN 80 and water and is maintained at pH 7.2. In another aspect, the pharmaceutical composition consists of 50 mg/mL of SEQ ID NO:1, sodium phosphate, trehalose, mannitol, TWEEN 80 and water and is maintained at pH 7.2. Pharmaceutical composition of the invention may comprise albiglutide, 2.8% mannitol, 4.2% trehalose dihydrate, 0.01% polysorbate 80, and phosphate buffer at pH 7.2.

In one embodiment, pharmaceutical compositions of the present invention are administered subcutaneously. In one aspect, the pharmaceutical composition is administered as a subcutaneous injection selected from the group of at least one 0.32 mL injection, at least one 0.65 mL injection, and at least one 1.0 mL injection.

In one embodiment the pharmaceutical composition reduces HbA1c and/or reduces serum glucose in a human with renal impairment. In some aspects the human has received metformin previous to receiving a pharmaceutical composition of the invention. In other aspects the pharmaceutical composition is administered as monotherapy.

In one aspect the pharmaceutical composition comprising a GLP-1 agonist is administered to said human at an initial dose of 30 mg of said GLP-1 agonist and subsequently titrated up to 50 mg of said GLP-1 agonist.

In another embodiment, methods are provided for treating type 2 diabetes in a human wherein said human has renal impairment comprising administering to said human a subcutaneous injection of a pharmaceutical composition comprising albiglutide. In one aspect, the pharmaceutical composition comprising a albiglutide is administered to said human at an initial dose of 30 mg of albiglutide per week. In another aspect, the pharmaceutical composition comprising a albiglutide is administered to said human at an initial dose of 30 mg of albiglutide per week and subsequently titrated up to 50 mg of albiglutide per week.

Pharmaceutical compositions of the present invention can be administered subcutaneously. Pharmaceutical composition can be administered as a subcutaneous injection selected from the group of: at least one 0.32 mL injection, at least one 0.65 mL injection, and at least one 1.0 mL injection. In some aspects, the pharmaceutical composition is co-administered in two injections, which may be the same dose or may be different doses of the same pharmaceutical composition. The pharmaceutical compositions of the present invention may be administered at the same or different injection sites. Subcutaneous injections of the invention may be administered as single injections, meaning the entire dose is administered as a single shot, wherein the entire volume of the shot is administered all at once. A single shot differs from a continuous administration which may be administered over several minutes and/or hours and/or days. Single injections may be administered multiple times, meaning as a single shot once daily, weekly, every two weeks, monthly and/or more.

In another aspect, the pharmaceutical composition reduces A1C in said human and/or serum glucose in said human with renal impairment. The serum half-life of said at least one polypeptide having GLP-1 activity, for example SEQ ID NO:1, is about 5 days. In another aspect, the human with renal impairment has a disease associated with elevated glucose levels which may include hyperglycemia, diabetes, type 2 diabetes mellitus. In yet another aspect, the pharmaceutical compositions of the present invention cause weight loss in a human when administered to a human.

In another embodiment, pharmaceutical compositions of the present invention are administered as monotherapy. In yet another embodiment, the pharmaceutical compositions is co-administered with at least one second hypoglycemic agent. A second hypoglycemic agent may be selected from: a GLP-1 agonist, incretin hormone, incretin mimetic, agent to increase insulin secretion, sulfonylurea, meglitinide, acetohexamide, chlorpropamide, tolazamide, glipizide, gliclazide, glibenclamide (glyburide), gliquidone, glimepiride, agent to inhibit GLP-1 break down, DPP-IV inhibitor, agent to increase glucose utilization, glitazones, thiazolidinediones, rosiglitazone, pioglitazone, pPAR agonists, agent to reduce hepatic glucose production, metformin, agent to delay glucose absorption, α-glucosidase inhibitor, insulin glargine and/or insulin. In some aspects, the second hypoglycemic agent is metformin. In some aspects, the second hypoglycemic agent is piaglitizone.

In some aspects of the present invention, albiglutide is administered to said human with type 2 diabetes mellitus and renal impairment at a dose of 30 mg once weekly (QW). In some aspects of the present invention, albiglutide is administered to said human with type 2 diabetes mellitus and renal impairment at an initial dose of 30 mg (QW) and subsequently titrated up to 50 mg (QW). The skilled artisan will understand that pharmaceutical compositions can be administered to humans who are no longer responding to their current therapy. That is, a subject may have a wash-out period from current therapy while concurrently or sequentially starting therapy with a pharmaceutical composition of the present invention.

In one embodiment, methods are provided for providing glycemic control in a human with type 2 diabetes mellitus and renal impairment, comprising administering to said human a pharmaceutical composition comprising between and including about 30 mg of albiglutide to about 50 mg of albiglutide per week. In one aspect, the composition is administered by subcutaneous injection.

As is understood in the art, various methods may be employed to collect, measure and assess pharmacokinetic data such as active compound concentration in blood, plasma and/or other tissue. As is also understood in the art, various methods may be employed to collect, measure and assess various pharmacodynamic data such as, but not limited to, renal clearance, glucose, insulin, C peptide, glucagon and other biomarker levels in blood and/or plasma and/or other tissue.

A skilled artisan will understand the various methods for measuring and calculating the pharmacokinetic (for example, but not limited to, Cmax, AUC, Tmax, serum half-life) and pharmacodynamic (for example, but not limited to, serum and/or plasma blood glucose levels and/or HbA1c levels) parameters described herein. Furthermore, the skilled artisan will understand the various methods for making statistical comparisons (for example, but not limited to, comparisons of change from baseline to post-treatment and/or comparisons among treatment groups) and/or analysis of the pharmacokinetic and pharmacodynamic parameters described herein. Furthermore, the skilled artisan will understand and be able to employ various other methods for collecting and analyzing pharmacokinetic, pharmacodynamic and other clinical data.

### EXAMPLES

The following examples illustrate various non-limiting aspects of this invention. For the following examples, unless noted otherwise, SEQ ID NO.:1 also referred to herein as Albiglutide (ALB) was formulated as 50 mg/mL from a lyophilized form comprising 2.8% mannitol, 4.2% trehalose dihydrate, 0.01% polysorbate 80, 10 mM phosphate buffer at pH 7.2. Compositions comprising SEQ ID NO.:1 were diluted with water for injection as necessary for respective dosing.

### Example 1

### Once Weekly (QW) Glucagon-Like Peptide 1 Receptor Agonist Albiglutide (Albi) Vs Sitagliptin (Sita) for Patients (Patients) with Type 2 Diabetes (T2D) with Renal Impairment (RI): Week 26 Results

Therapies for type 2 diabetes mellitus (T2DM) with renal impairment (RI) are limited and may require dose adjustment. This 52-week randomized, double blind, active controlled, Phase III parallel group study examined efficacy/safety of QW Albi injections (30 mg uptitrated to 50 mg if needed) vs daily Sita in patients with T2D and RI (eGFR≧15 and <90 mL/min/1.73 m²) and A1C 7-10% on lifestyle (11% of subjects) or metformin (Met), thiazoldinedone (TZD), sulfonylureas (SU), or any combination (89% of patients). Sita was dosed by RI degree per prescribing information; Albi dose did not require modification. Primary endpoint was A1C change at week 26 for Albi vs Sita (noninferiority with subsequent superiority analysis). Eligible subjects were men or non pregnant, nonlactating women, 18 years of age or older, with a historical diagnosis of T2DM who were experiencing inadequate glycemic control on their current regimen of diet and exercise or their antidiabetic therapy of metformin, TZD, SU, or any combination of these OAD medications. Subjects had a fasting C-peptide at least 0.8 ng/mL (≧0.26 nmol/L); hemoglobin at least 10 g/dL (≧100 g/L) for male subjects and at least 9 g/dL (≧90 g/L) for female subjects; and GFR at least 15 mL/min/1.73 m² and less than 90 mL/min/1.73 m² using the Modification of Diet in Renal Disease (MDRD) formula.

Demographics matched across groups, including degree of RI (mild, moderate, severe); mean age 63 years, weight 83 kg, A1C 8.2%. A1C decreased in both groups: −0.83% for Albi (n=246) and −0.52% for Sita (n=240) at week 26; treatment difference −0.32% (95% CI −0.49%, −0.15%). Albi was superior to Sita (P=0.0003). Mean A1C change was greater with Albi vs Sita regardless of degree of RI. 35% of Albi patients uptitrated to 50 mg. A1C<7% was achieved by 42.6% of Albi. 30.5% of Sita patients (p=0.006) (See Table 1). FPG (−25.6 mg/dL vs −3.9 mg/dL, P<0.0001) and weight change (−0.8 kg vs −0.2 kg, P=0.0281) favored Albi.

Adverse Event (AE) rates for Albi/Sita were low in both groups: nausea: 4.8%/2.8%; diarrhea: 8.8%/6.1%; vomiting: 1.6%/0.8%. Hypoglycemia rates were 20.5% (0 severe) for Albi (94% on SU) vs 13.4% (2 severe) for Sita (91% on SU). Injection site reaction rates: Albi 6.8%; Sita 2.8%. Anti-Albi antibody rate was 2.8% (0% neutralizing) (See Table 2).

Thus, in T2D with RI, QW Albi had superior A1C reductions at week 26 vs Sita without requiring dose adjustment. Both agents were well tolerated.

TABLE 1
Change From Baseline in A1C at Week 26 by Severity of Renal Impairment

| | Albiglutide | | | | Sitagliptin | | | |
|---|---|---|---|---|---|---|---|---|
| | Mild | Moderate | Severe | Any | Mild | Moderate | Severe | Any |
| Renal impairment severity | (60-89) | (30-59) | (15-29) | (15-89) | (60-89) | (30-59)) | (15-29) | (15-89) |
| (eGFR range, mL/min/1.73 m2) | n = 125 | n = 98 | n = 19 | n = 246 | n = 122 | n = 99 | n = 15 | n = 240 |
| Baseline A1C, mean, % | 7.96 | 8.26 | 8.05 | 8.08 | 8.16 | 8.28 | 8.32 | 8.22 |
| Week 26 A1C, mean, % | 7.23 | 7.37 | 6.97 | 7.27 | 7.50 | 7.91 | 7.67 | 7.68 |
| Change from baseline A1C, | −0.72 | −0.88 | −1.08 | −0.82 | −0.66 | −0.37 | −0.65 | −0.54 |
| mean (SD), % | (0.81) | (1.00) | (0.91) | (0.90) | (0.88) | (1.33) | (1.24) | (1.12) |
| Model-adjusted change from | −0.80 | −0.83 | −1.08 | −0.83 | −0.67 | −0.31 | −0.61 | −0.52 |
| baseline A1C, a LS mean (SE), % | (0.09) | (0.10) | (0.22) | (0.06) | (0.09) | (0.10) | (0.25) | (0.06) |
| Difference of LS means from | −0.13 | −0.53 | −0.47 | 0.32 | | | | |
| sitagliptin (95% CI), a % | (−0.37, 0.11) | (−0.80, −0.26) | (−1.12, 0.18) | (−0.49, −0.15) | | | | |
| Superiority P value | | | | .0003 | | | | |

Based on ANCOVA model accounting for Tx, baseline A1C, renal impairment, prior MI history, age and region.

TABLE 2
Most Common On-therapy Adverse Events (>5% in Either Arm) Through
Week 26 (Safety Population)

n (%)

| | Albiglutide | Sitagliptin |
|---|---|---|
| Adverse Event | (N = 249) | (N = 246) |
| Diarrhea | 22 (8.8) | 15 (6.1) |
| Constipation | 15 (6.0) | 5 (2.0) |
| Nasopharyngitis | 13 (5.2) | 16 (6.5) |
| Hypertension | 10 (4.0) | 15 (6.1) |
| Upper respiratory tract infection | 9 (3.6) | 16 (6.5) |
| Urinary tract infection | 8 (3.2) | 15 (6.1) |

In both treatment groups, AE rates were low and diarrhea was the most common event.

### Conclusion:

Once-weekly fixed-dose albiglutide resulted in superior glycemic control as well as a modest reduction in body weight compared with dose-adjusted sitagliptin in patients with inadequate glycemic control and varying degrees of renal impairment. Albiglutide was generally well-tolerated. Albiglutide will be a useful treatment addition for patients with renal impairment and type 2 diabetes without the need for dose adjustment.

### Example 2

### Effect of Renal Impairment on the Pharmacokinetics, Efficacy and Safety of Albiglutide

Chronic kidney disease is frequently present in patients with type 2 diabetes mellitus (T2DM) and new therapeutic options in this patient subpopulation are needed.

### Objectives:

Assess the effect of renal impairment on the pharmacokinetics (PK), efficacy, and safety of albiglutide in single and multiple dose studies.

### Methods:

PK, safety, and efficacy of once weekly albiglutide in patients with T2DM was assessed from a single dose (30 mg) nonrandomized, open-label study (N=41) including subjects with normal and varying degrees of renal impairment, including hemodialysis, and in 4 Phase 3 randomized, double-blind (one open-label), active or placebo-controlled multiple dose studies. The pooled analysis of the latter 4 studies (N=1113) was part of the population PK analysis which included normal subjects and those with varying degrees of renal impairment (mild, moderate, severe) treated with albiglutide (30 to 50 mg) out to primary endpoints of 26 to 52 weeks.

### Results:

Single dose PK showed AUC ratios (and 90% CI) of 1.32 (0.96, 1.80), 1.39 (1.03, 1.89) and 0.99 (0.63, 1.57) for the moderate, severe and hemodialysis groups respectively relative to the normal group. Results indicate that modest increases in plasma concentration of albiglutide were observed with the severity of renal impairment. There was a trend for more glycemic lowering as eGFR decreased. The severe group had a higher frequency of GI events compared to patients with mild or moderate renal impairment and there was a trend towards higher amounts of certain GI events (constipation and diarrhea) across the spectrum of renal impairment.

### Conclusion:

The totality of the PK, efficacy and safety data indicate that albiglutide has a favorable benefit:risk in patients with T2DM and any degree of renal impairment and the need for a dose adjustment is not expected.

We report here on the overall effect of renal impairment on the PK, efficacy, and safety of albiglutide from both a Phase 1 single-dose study in patients with T2DM and varying degrees of renal impairment, including those requiring hemodialysis, as well as patients included in a Phase 3 population PK analysis of 4 randomized and well controlled studies (including a dedicated renal study in patients with T2DM who had varying degrees of renal impairment). ¹⁰⁻¹³

### Methods

### Single-Dose Pharmacokinetics

A Phase 1, open-label, nonrandomized study evaluated the pharmacokinetics of a single 30 mg subcutaneous dose of albiglutide in subjects with T2DM and normal or varying degrees of renal impairment. Eligible subjects were men and women from 30 to 75 years of age (inclusive) with T2DM and HbA1c between ≧6.0% and ≦10.5%. Exclusion criteria included: any clinically relevant disease history that could interfere with the study interpretation; systolic blood pressure <90 mm Hg or diastolic blood pressure <45 mm Hg; and use of any drugs known to significantly inhibit or induce cytochrome P450 enzymes within 7 days before screening or current treatment with a sulfonylurea or GLP-1R agonist. Participants on hemodialysis were on stable treatment for at least 3 months before screening and had serum albumin ≧2.5 g/L and hemoglobin ≧8 g/dL.

Venous blood samples for determination of albiglutide were drawn on Day 1 prior to dosing and 6 hours after dosing and on Days 2, 3, 5, 10, 14, 21, 29, and 42 after administration of albiglutide (30 mg subcutaneous). In subjects undergoing hemodialysis, Day 1 was planned to be the day before a scheduled hemodialysis day (Day 2). Following blood sampling on Day 1 (before dosing and 6 hours after dosing) blood samples were taken before and after each dialysis procedure over the next 7 weeks. For all subjects, each blood sample was about 3 mL and drawn into an EDTA collection tube, immediately placed on crushed ice and water, and centrifuged for 10 minutes at 1,500 g at 4° C. Approximately 1 mL of plasma was transferred to a sterile 1.8-mL tube (Nunc® CryoTube®, Sigma-Aldrich) and stored at −70° C. until assaying.

Albiglutide PK samples collected in participants undergoing hemodialysis also were analyzed to assess predialysis and postdialysis changes in albumin concentrations.

### Pooled Analysis—Population Pharmacokinetics, Safety, and Efficacy

Patients with T2DM were enrolled in 3 similarly designed Phase 3 studies ¹¹⁻¹³ (Harmony 2, Harmony 4 and Harmony 5) and one Phase 3 study in patients with varying degrees of renal impairment (Harmony 8). ¹⁰ These studies were randomized, double-blind (one open-label), active- (sitagliptin, pioglitazone, or glimepiride) or placebo-controlled, parallel-group, multicenter trials in which albiglutide (30 to 50 mg) was administered once weekly. In all 4 studies, men and nonpregnant women who were ≧18 years of age with a historical diagnosis of T2DM, and who were inadequately controlled (HbA1c≧7.0% and ≦10.0%) on diet and exercise and/or their current regimen of anti-diabetic agents (ie, metformin, thiazolidinedione, sulfonylurea, or any combination of these oral anti-diabetic medications) were eligible to enroll. Additional eligibility criteria included fasting C peptide of ≧0.8 ng/mL (≧0.26 nmol/L) and BMI between 20 and 45 kg/m². Harmony 8, the study in CKD patients, required estimated GFR≧15 and <90 mL/min/1.73 m², whereas the other 3 studies required estimated GFR>60 mL/min/1.73 m². Exclusion criteria for participants in all 4 studies included malignant disease (except squamous cell or basal cell carcinoma); a history of diabetic gastroparesis; current ongoing symptomatic biliary disease or history of pancreatitis; significant GI surgery or surgeries thought to significantly affect upper GI function; recent (within predefined time scales) clinically significant cardiovascular and/or cerebrovascular disease; a history of human immunodeficiency virus infection; and acute symptomatic hepatitis B or C infection. Patients who met additional exclusion criteria, including requirements for levels of total bilirubin, alanine aminotransferase, aspartate aminotransferase, amylase, lipase, fasting triglycerides, and hemoglobin were also excluded.

During each Phase 3 study, 4 blood samples for population pharmacokinetic evaluation were obtained. In the study of patients with CKD, 2 predose samples were obtained at weeks 8 and 16 and 2 postdose samples were obtained at least 2 days after dosing between weeks 8 and 10 and weeks 16 and 20. In the other 3 studies, 2 predose samples were obtained at weeks 8 and 24 and 2 postdose samples were obtained at least 2 days after dosing between weeks 8 and 10 and weeks 24 and 28. The blood collection and storage procedure were the same as described above.

Efficacy (HbA1c, fasting plasma glucose [FPG] and body weight) was assessed in each of the 4 studies by change from baseline to the primary endpoint (week 26 to week 52) using last observation carried forward for missing data. Rescue medications were allowed in subjects with persistent hyperglycemia (REF), however, efficacy data obtained after hyperglycemic rescue were not included and replaced with pre-rescue values for efficacy evaluation. Safety was assessed by monitoring the incidence of treatment emergent adverse events throughout the studies.

### Albiglutide Assay Procedures

Plasma samples were analyzed for albiglutide by using a validated enzyme-linked immunosorbent assay (ELISA) and were diluted 100-fold with sample buffer before analysis. Albiglutide was captured using a rabbit anti-human GLP-1 (7-36) amide and detected using a rabbit anti-HSA conjugated to biotin. ELISA experiments were performed using the Furoninc® 96 well plate (Nunc, cat. #437796) with the following steps. 1) 100 ml, antibody coating solution of 5.0 μg/mL anti-GLP-1 (7-36) Amide (human) IgG (Peninsula Laboratories, cat. # T-4111) in 50 mM sodium carbonate-bicarbonate buffer pH 9.4 was added to each well, sealed with aseptic sealing tape and incubated for 24 hours (±12 hours) at nominally 4° C. The microtiter plate was washed 5 times with 300 mL wash buffer (10 mM Sodium Phosphate, 150 mM NaCl, 0.05% Tween 20, pH 7.5) using plate washer; 2) 200 mL blocking buffer of SuperBlock in PBS (SuperBlock (Pierce, cat. #37515) in PBS, 5.0% Rabbit Plasma) was added to each well, sealed and incubated with constant shaking for approximately 1.5 hour (±30 minutes) at ambient room temperature and the microtiter plate washed; 3) 100 mL of either standards, quality controls (QCs) or samples were added per well, sealed, incubated and washed; 4) 100 mL of Detection Antibody solution of Biotin Labelled Rabbit polyclonal to Human Serum Albumin (NOVUS Biologicals, cat. # NB 100-1674) was added per well, sealed, incubated and washed; 5) 100 mL of Reporter Tag solution (Streptavidin-HRPO Conjugate, Caltag, cat. # SA1007) was added per well, sealed, incubated and washed; 6) 100 mL of Development Substrate Pico (SuperSignal ELISA, Pierce, cat. #37070) was added, incubated and the plate read using plate reader (Wallac 1420 Victor Multilabel Counter, Perkin Elmer Life Sciences, Boston, Mass.). Sample concentrations were determined by interpolation from the standard curve, which was fitted using a weighted (1/x), 4-parameter logistic equation. The validated range of this assay (based on 10 μL of human plasma) is 50 ng/mL to 1,500 ng/mL.

During the single dose study and 3 of the phase 3 trials (Harmony 2, 4 and 5), the assay process was modified from manual pipetting of samples to robotic pipetting. It was determined that manual pipetting systematically resulted in underestimation of albiglutide concentrations by introduction of a time-dependent incubation period, according to the duration of the manual sample handling procedure. Confirmatory evidence was seen in an incurred sample reanalysis ¹⁴ of 32 manually pipetted samples from the single dose study. An average difference of 1.6-fold lower was seen in albiglutide concentrations between manual versus robotic pipetting and therefore samples affected by the manual pipetting underestimation were corrected by the incurred sample reanalysis factor for the single dose study.

### Pharmacokinetic and Statistical Analysis

### Single Dose Study

Pharmacokinetic calculations were based on actual sampling collection times. All statistical analyses of pharmacokinetic parameters were performed using SAS Version 9.1 or later (SAS Institute, Inc., Cary, N.C.). The Cₘₐₓ and AUC_{(0-∞)} for albiglutide were compared between participants with normal renal function and participants with moderate and severe renal impairment and those requiring hemodialysis. The comparisons were performed using an analysis of variance on natural log-transformed Cₘₐₓ and AUC_{(0-∞)} with cohort as a fixed effect. The geometric least squares mean ratios of Cₘₐₓ and AUC_{(0-∞)} on the original scale were calculated by exponentiation of the least squares mean differences of the natural log-transformed PK parameters. Similarly, the associated 90% confidence interval of the ratios was calculated by exponentiation of lower and upper limits of a 90% confidence interval of the mean differences.

Plasma concentrations of albiglutide were compared before and after hemodialysis. No formal statistical analysis of these concentrations was performed. After PK concentration data were analyzed, the samples for participants undergoing hemodialysis were submitted for analysis of albumin. Predialysis and postdialysis fluctuations in albiglutide concentrations were compared graphically with albumin fluctuations.

### Phase 3 Clinical Studies (Population PK Analysis)

Population PK analyses were performed using NONMEM (Version 7.1.2), PDx-Pop (Version 4.20) and Intel Visual Fortran Compiler (Version 12) on Microsoft Windows XP Professional. For optimal model selection, the NONMEM diagnostic plots as appropriate, during the preliminary runs were evaluated at each stage of model development for appropriateness of the initial estimates and final estimates of model parameters. The standard criteria of change in the minimum objective function value (OFV) and review of standard diagnostics were conducted. Potential covariates affecting the PK of albiglutide were explored, including eGFR, body weight, race, and concomitant use of insulin. A one-compartment PK model with first order absorption and elimination processes was selected to describe the PK of albiglutide, parameterized in terms of apparent clearance, apparent volume of distribution and absorption rate (CL/F, V/F, and ka respectively).

To evaluate efficacy and tolerability in different renal population, efficacy and tolerability data from patients with pop PK sample across 4 Phase 3 studies were extracted. Descriptive analysis were conducted up to primary endpoint (week 52 for Harmony 2, Harmony 4 and Harmony 5 and week 26 for study Harmony 8). Baseline demographics are summarized by renal impairment severity (normal, mild, moderate and severe). Efficacy parameters such as change from baseline of HbA1c, FPG, weight are also summarized by renal impairment severity. LOCF (Last Observation Carried Forward) approach is used in efficacy evaluation. Except for hypoglycemia, tolerability (AE, SAE and AE cause withdraw) were summarized by renal impairment severity for all data up to primary endpoint including post-rescue data. Hypoglycemia events (severe and documented symptomatic) were evaluated by renal impairment severity only include pre-rescue data.

### Study Ethics

This study was conducted in accordance with ICF GCP and the Declaration of Helsinki, and all patients provided written informed consent before they participated in this study.

### Results

### Single Dose Pharmacokinetics Study

Baseline demographics and subject characteristics are presented in Table 3.

TABLE 3
Subject demographics and baseline characteristics in Single Dose Study
(Safety Population)

| | Chronic Kidney Disease Cohort | | | |
|---|---|---|---|---|
| | | | | Hemo- |
| | Normal | Moderate | Severe | dialysis |
| | n = 10 | n = 11 | n = 10 | n = 10 |
| Age, yrs, mean | 50.5 (9.7) | 62.7 (6.1) | 61.9 (9.8) | 52.0 (9.6) |
| (SD) | | | | |
| Gender, M:F, n | 5:5 | 8:3 | 4:6 | 8:2 |
| Race, n | | | | |
| White/Caucasian | 7 | 8 | 4 | 3 |
| African | 3 | 2 | 6 | 7 |
| American/ | | | | |
| African\ | | | | |
| Asian | 0 | 1 | 1 | 0 |
| Duration of | 8.3 (4.9) | 11.0 (7.6) | 21.3 (12.3) | 18.3 (6.7) |
| diabetes, years, | | | | |
| mean (SD) | | | | |
| BMI (kg/m²) | 31.7 (4.9) | 30.7 (6.0) | 32.5 (5.4) | 29.9 (5.1) |
| mean (SD) | | | | |
| Estimated GFR, | 118 (22.7)) | 39.1 (5.9) | 21.3 (4.9) | 9.2 (2.9) |
| mL/min/1.73 m² | | | | |
| Mean (SD) | | | | |

GFR, glomerular filtration rate;
min, minimum;
max, maximum;
Normal = estimated GFR >80 mL/min/1.73 m²;
moderate = GFR ≧30 and <50 mL/min/1.73 m²;
severe = estimated GFR ≧15 and <30 mL/min/1.73 m²;
hemodialysis = requiring hemodialysis.

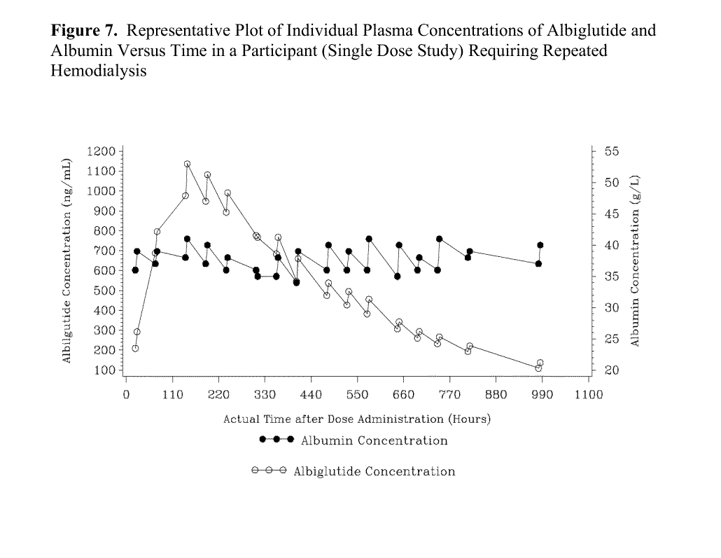

Cohorts with varying degrees of renal impairment were reasonably matched for age, gender, and BMI. The results from this study showed AUC ratios (and 90% CI) of 1.32 (0.96, 1.80), 1.39 (1.03, 1.89) and 0.99 (0.63, 1.57) for the moderate, severe and hemodialysis groups respectively relative to the normal group (Table 4). The subjects undergoing hemodialysis demonstrated a clear trend for increased exposure in the immediate post-dialysis period (FIG. 7). Based on a similar change in albumin concentration, this concentrating effect was due to the removal of water during the hemodialysis procedure rather than a direct effect on albiglutide pharmacokinetics.

TABLE 4
Single Dose Pharmacokinetic Comparison of Albiglutide: Renal
Impairment to Normal

| Parameter | Comparison | Ratio | 90% CI |
|---|---|---|---|
| AUC | Moderate:Normal | 1.32 | (0.96, 1.80) |
| | Severe:Normal | 1.39 | (1.03, 1.89) |
| | Hemodialysis:Normal | 0.99 | (0.63, 1.57) |
| Cmax | Moderate:Normal | 1.21 | (0.90, 1.63) |
| | Severe:Normal | 1.23 | (0.91, 1.67) |
| | Hemodialysis:Normal | 1.11 | (0.72, 1.73) |

Normal = estimated glomerular filtration rate >80 mL/min/1.73 m²;
moderate = glomerular filtration rate ≧30 mL/min/1.73 m²;
severe = glomerular filtration rate <30 mL/min/1.73 m²;
hemodialysis = participants requiring hemodialysis.

Regardless of the level of renal impairment, the median time of peak albiglutide concentrations (Tₘₐₓ) was comparable (88-96 h after dosing). The half-life was slightly greater in participants with impaired renal function (geometric mean of 141-145 h) compared with participants with normal renal function (geometric mean of 122 h).

A summary of adverse events are presented in Table 5.

TABLE 5
Summary of Adverse Events in Single Dose Study (Reported by ≧2
Participants)

| | Participants Combined by Level | | | |
|---|---|---|---|---|
| | of Renal Impairment* | | | |
| | Normal | Moderate | Severe | Hemodialysis |
| Preferred Term | (n = 10) | (n = 11) | (n = 10) | (n = 10) |
| Any event, n (%) | 8 (80) | 3 (27.3) | 4 (40) | 8 (80) |
| Total no. of events, n | 8 | 4 | 8 | 38 |
| Nausea | 0 | 0 | 0 | 5 (50) |
| Vomiting | 0 | 0 | 0 | 5 (50) |
| Headache | 0 | 0 | 1 (10) | 3 (30) |
| Dizziness | 0 | 0 | 0 | 2 (20) |
| Fatigue | 0 | 0 | 0 | 2 (20) |
| Hypoglycemia, | 0 | 0 | 2 (20) | 1 (10) |
| Documented | | | | |
| Symptomatic** | | | | |

*Normal = estimated glomerular filtration rate >80 mL/min/1.73 m²; moderate = glomerular filtration rate ≧30 mL/min/1.73 m²; severe = glomerular filtration rate <30 mL/min/1.73 m²; hemodialysis = participants requiring hemodialysis.
**ADA Criteria: Documented symptomatic-plasma glucose concentration ≦70 mg/dL and presence of hypoglycemic symptoms

A higher incidence of AEs was seen in the subjects on hemodialysis. The majority of AEs were mild or moderate in intensity and no clinically meaningful trends in clinical laboratory evaluations, electrocardiograms, or vital signs were seen. One insulin-treated subject in the hemodialysis group experienced a hypoglycemic SAE and one subject in the moderate group withdrew consent.

### Population Pharmacokinetics—Phase 3 Pooled Analysis

A total of 1,113 patients treated with albiglutide were included in these analyses. Demographic and baseline characteristics of these patients are presented in Table 6.

TABLE 6
Subject Baseline Demographics and Characteristics in the Phase 3 Pooled
Analysis*

| | Chronic Kidney Disease Cohort | | | | | | | |
|---|---|---|---|---|---|---|---|---|
| | Normal | | Mild | | Moderate | | Severe | |
| | (≧90) | | (≧60 to <90) | | (≧30 to <60) | | (≧15 to <30) | |
| eGFR category, mL/min/1.73 m² | n = 289 | | n = 634 | | n = 171 | | n = 19 | |
| eGFR, mL/min/1.73 m², mean | 106 | (0.75) | 75.9 | (0.33) | 50.0 | (0.6) | 23.7 | (0.9) |
| (SEM) | | | | | | | | |
| Age (years), mean (SEM) | 50.0 | (0.6) | 57.8 | (0.4) | 64.4 | (0.7) | 60.4 | (1.9) |
| Gender, Male, n (%) | 142 | (49.1) | 354 | (55.8) | 94 | (55.0) | 9 | (47.4) |
| Race, n (%) | | | | | | | | |
| White | 169 | (58.5) | 458 | (72.3) | 102 | (59.6) | 5 | (26.3) |
| African American/African | 83 | (28.7) | 89 | (14.0) | 22 | (12.9) | 3 | (15.8) |
| Asian | 16 | (5.6) | 69 | (10.9) | 35 | (20.5) | 7 | (36.9) |
| Duration of diabetes (years), | 7.2 | (0.4) | 8.3 | (0.3) | 10.4 | (0.6) | 15.8 | (1.9) |
| mean (SEM) | | | | | | | | |
| Baseline HbA1c (%), mean | 8.4 | (0.06) | 8.2 | (0.04) | 8.1 | (0.07) | 8.1 | (0.17) |
| (SEM) | | | | | | | | |
| Baseline FPG, mg/dL, mean | 169 | (3.1) | 172 | (2.0) | 163 | (4.7) | 177 | (15.2) |
| (SEM) | | | | | | | | |
| Body weight (kg), mean (SEM) | 91.6 | (1.2) | 93.3 | (0.8) | 90.3 | (1.6) | 81.2 | (4.9) |
| BMI (kg/m²) mean (SEM) | 33.2 | (0.3) | 32.9 | (0.2) | 32.3 | (0.5) | 31.1 | (1.4) |
| Concomitant antidiabetic | | | | | | | | |
| medications | | | | | | | | |
| Insulin | 0 | | 5 | (0.8) | 14 | (8.2) | 3 | (15.8) |
| Metformin | 237 | (82.0) | 421 | (66.4) | 65 | (38.0) | 0 | |
| Sulfonylurea | 192 | (66.4) | 406 | (64.0) | 133 | (77.8) | 14 | (73.7) |
| Thiazolidinediione | 0 | | 2 | (0.3) | 1 | (0.6) | 0 | |

*Analysis includes an integration of four Phase 3 studies (Harmony 2, 4, 5, and 8) in subjects treated with albiglutide

Gender, BMI, and baseline glycemic control was generally similar between groups. The majority of subjects were white except for the severe group which had the largest percentage of Asians. Patients in the renal impaired groups were older than subjects with normal renal function and the severe group had the longest duration of diabetes, the higher percentage of subjects on sulphonylureas, and the smallest body weight.

The final population pharmacokinetic model revealed that the apparent clearance (CL/F) of albiglutide was a function of body weight, eGFR, African American/African heritage, and concomitant administration of insulin. The relationship between these covariates and CL/F was described by the following equation:

CL/F (mL/hr)=66.6·(BW/90.7)^{0.932}·(eGFR/77.9)^{0.219}·0.778 (if African American/African heritage)·1.22 (if currently receiving insulin)

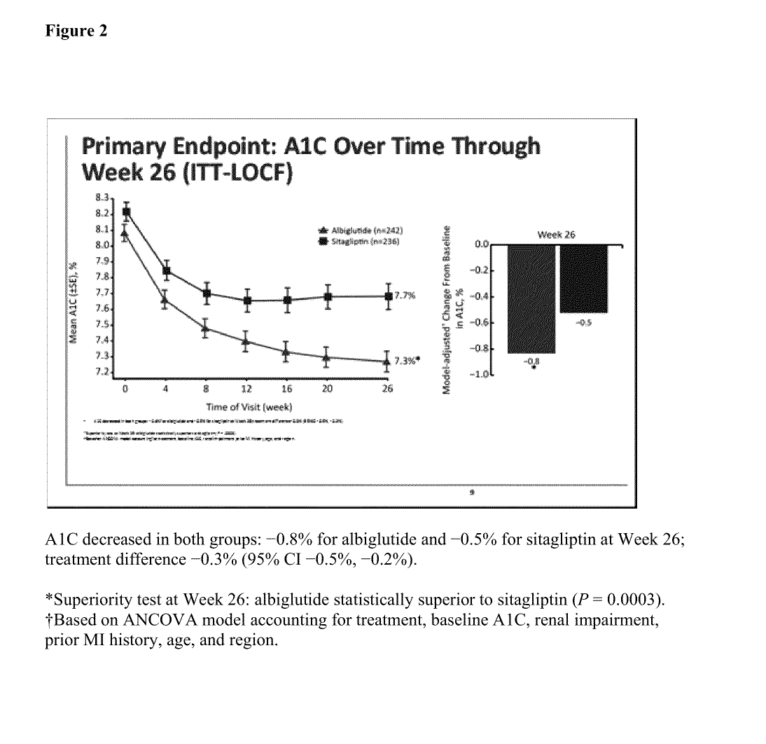

Based on this model, the effect of eGFR on albiglutide clearance was modest (FIG. 2).

For example, compared to a person with eGFR around 80 mL/min/1.73 m² (approximate median value of the pooled PK population), albiglutide clearance in a typical patient with an eGFR of 30 mL/min/1.73 m² (severe renal impairment) would be decreased by about 19% while in a patient with an eGFR of 50 mL/min/1.73 m² (mild renal impairment) the decrease would be about 9%. Patients with eGFR as low as 15 mL/min/1.73 m² would have a decrease in clearance of up to 30%.

### Efficacy—Pooled Analysis

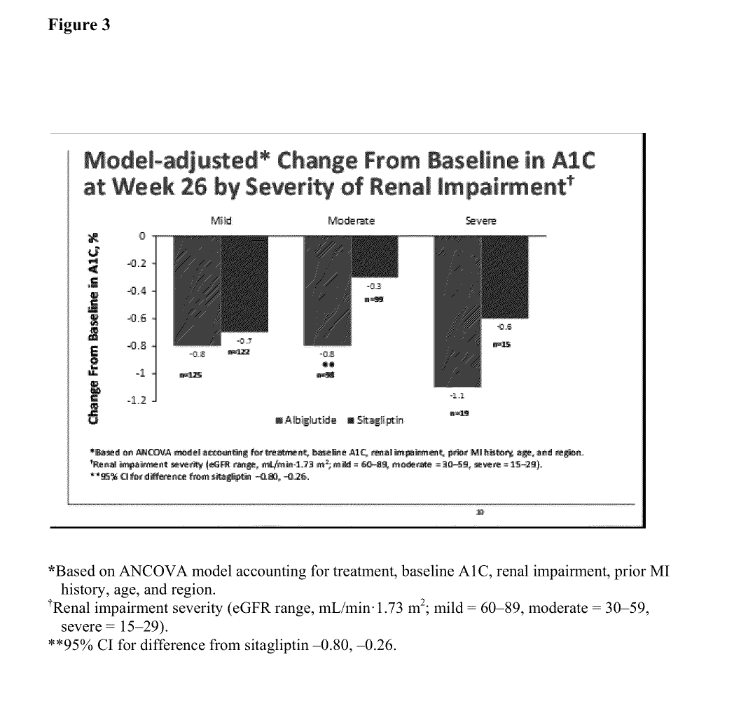

Patients within the normal and mild, moderate and severe renal impairment categories had a range of GFRs and were integrated from 4 different clinical studies, each of which had different subject inclusions, so the efficacy data was not presented as means but rather in a scatter plot (FIG. 3). The treatment effect of albiglutide, as measured by HbA1c and FPG, showed a trend towards greater reductions as GFR decreased. Patients in all groups showed a modest mean loss in body weight which ranged from −0.2 kg to −1.6 kg.

### Safety—Pooled Analysis

The safety population consisted of 286, 633, 175, and 19 subjects in the normal, mild, moderate, and severe renal impairment groups, respectively. Given the low number of subjects in the severe group, interpretations for this group need to be well considered. Overall, any AE and serious AEs occurred at similar rates in the normal and the mild and moderate impaired subjects while the severe group had a lower percentage (Table 7). Withdrawal rates from AEs were generally low but higher in the renally impaired groups (Table 3). The most common on-therapy AEs are listed in Table 3. Anemia, renal impairment, and peripheral edema occurred more frequently in the severely impaired group, although there was no clear trend when looking across groups from normal to moderately impaired. Trends towards higher amounts of constipation and diarrhea with worsening renal function was observed but it was not clearly seen for nausea/vomiting (percentages were 8.0/2.1, 9.5/3.2, 7.4/2.9, and 16/5.3 for the normal, mild, moderate, and severe groups, respectively, with only one subject in the severe group reporting one event of vomiting).

TABLE 7
Overall Safety Through Primary Endpoint in the Phase 3 Pooled Analysis
(Safety Population)*

| | Chronic Kidney Disease Cohort | | | |
|---|---|---|---|---|
| | Normal | Mild | Moderate | Severe |
| | n = 286 | n = 633 | n = 175 | n = 19 |
| | N (%) | N (%) | N (%) | N (%) |
| Overall Safety | | | | |
| Any AE | 232 (81) | 511 (81) | 139 (79) | 12 (63) |
| Serious AE | 17 (5.9) | 52 (8.2) | 17 (9.7) | 0 (0) |
| AEs leading to | 11 (3.8) | 37 (5.8) | 11 (6.3) | 2 (11) |
| withdrawal | | | | |

Most Common On-therapy Adverse Events
(>7.0% in normal, mild, or moderate groups or >2 patients in severe group

| | | | | |
|---|---|---|---|---|
| Injection site reaction | 32 (11) | 51 (8.1) | 12 (6.9) | 1 (5.3) |
| Upper Respiratory | 22 (7.7) | 59 (9.3) | 8 (4.6) | 1 (5.3) |
| Tract Infection | | | | |
| Nasopharyngitis | 19 (6.6) | 51 (8.1) | 14 (8.0) | 1 (5.3) |
| Anemia | 6 (2.1) | 15 (2.4) | 5 (2.9) | 4 (21) |
| Renal Impairment | 0 (0) | 0 (0) | 3 (1.7) | 4 (21) |
| Edema peripheral | 5 (1.7) | 21 (3.3) | 4 (2.3) | 3 (16) |

Gastrointestinal disorders system organ class (≧2 patients in severe group)

| | | | | |
|---|---|---|---|---|
| Any event | 76 (27) | 205 (32) | 58 (33) | 8 (42) |
| Diarrhea | 20 (7.0) | 55 (8.7) | 23 (13) | 4 (21) |
| Nausea | 23 (8.0) | 60 (9.5) | 13 (7.4) | 3 (16) |
| Constipation | 8 (2.8) | 25 (3.9) | 11 (6.3) | 2 (11) |
| Abdominal distention | 2 (0.7) | 5 (0.8) | 2 (1.1) | 2 (11) |

Pre-Rescue Hypoglycemia**

| | | | | |
|---|---|---|---|---|
| Documented | 44 (15) | 73 (12) | 28 (16) | 4 (21) |
| Symptomatic (overall) | | | | |
| With background SU | 44 (21) | 70 (17) | 28 (20) | 4 (29) |
| Without background SU | 0 (0) | 3 (1.4) | 0 (0) | 0 (0) |
| Severe (overall) | 1 (0.3) | 1 (0.2) | 0 (0) | 0 (0) |
| With background SU | 1 (0.3) | 1 (0.2) | 0 (0) | 0 (0) |
| Without background SU | 0 (0) | 0 (0) | 0 (0) | 0 (0) |

*Analysis includes an integration of four Phase 3 studies (Harmony 2, 4, 5, and 8) with primary endpoint from 26 to 52 weeks in subjects treated with albiglutide.
**ADA Criteria: Severe-event requiring another person to administer a resuscitative action; Documented symptomatic-plasma glucose concentration ≦70 mg/dL and presence of hypoglycemic symptoms

The percentage of subjects in the albiglutide group who experienced a documented symptomatic hypoglycemic event was generally comparable among groups but numerically highest in the severe group, severe events were reported in 2 subjects (normal, mild renal impairment) with background SU use (Table 7). Nearly all subjects who had an event were on a background sulphonyurea medication.

Cases of pancreatitis have been reported previously.¹⁰⁻¹³ Two of the albiglutide-treated patients (one with mild and one with moderate renal impairment) included in the pooled PK analysis were identified as having probable pancreatitis (i.e., with symptoms and lab elevations) that was at least possibly related to study drug. No patient developed thyroid cancer. The incidence of anti-albiglutide antibodies in the 4 trials included in the pooled analysis ranged from 2.8% to 8.1% and none of the antibodies were neutralizing. ¹⁰⁻¹³

### Discussion

The results of the analyses presented indicate that modest increases in plasma concentration of albiglutide were observed with the severity of renal impairment with a trend for more glycemic lowering as eGFR decreased while for safety the severe group had a higher frequency of GI events compared to patients with mild or moderate renal impairment and there was a trend towards higher amounts of certain GI events (constipation and diarrhea) across the spectrum of renal impairment.

Treatment options for T2DM patients with CKD are limited because of the influence of reduced filtration capacity on safety and efficacy of most of the available antidiabetes medications. ³⁻⁵ Treatment guidelines for T2DM have positioned incretin-based therapies, such as albiglutide, as an alternative first-line therapy in certain clinical settings and as a prominent second-line therapy in most patients because of their safety profile, substantial effectiveness in improving glycemic control, and other positive attributes (ie, modest weight loss, low hypoglycemia rates). ¹⁴

The influence of CKD on the pharmacokinetics of currently available GLP-1 receptor agonists exenatide and liraglutide are not consistent. Linnebjerg and colleagues reported single-dose pharmacokinetics of exenatide (5 or 10 μg) in subjects with normal renal function (eGFR>80 mL/min), mild CKD (eGFR>50 and ≦80 mL/min), moderate CKD (eGFR>30 and ≦50 mL/min), and ESRD on hemodialysis. ¹⁵ They found no difference in dose and weight normalized AUC_{0-∞} in subjects with mild CKD compared with the subjects with normal renal function. However, exposure was 1.6-fold higher and 6.2-fold higher than in the control group in the moderate CKD and ESRD on dialysis cohorts, respectively. Importantly, they found that exenatide was well tolerated in the mild and moderate CKD cohorts, but was not in the ESRD group due to higher incidence of nausea and vomiting. Jacobsen et al. studied the pharmacokinetics of liraglutide in subjects with normal renal function and with varying degrees of CKD, including subjects with ESRD receiving continuous ambulatory peritoneal dialysis (but excluding subjects receiving hemodialysis). ¹⁶ In the latter study, each group with CKD (mild, eGFR>50 to ≦80 mL/min; moderate, eGRF>30 to ≦50 mL/min; severe, eGFR≦30 mL/min) demonstrated reduced exposure compared to the group with normal renal function, with no consistent trend with decreasing renal function.

The elimination of albiglutide likely occurs primarily through proteolysis, and based on its large molecular size, renal elimination of albiglutide should not contribute substantially to its clearance. The results of the single dose and the population pharmacokinetic studies presented here support this concept, as clearance of albiglutide was only modestly reduced as eGFR declined. The population pharmacokinetic modeling suggested that patients with eGFR in range of 15 mL/min/1.73 m² would experience about a 30% decrease in albiglutide clearance. Exposures of albiglutide in subjects requiring hemodialysis was in the range of the exposures in subjects with moderate and severe CKD. In subjects requiring hemodialysis, a clear trend for increased albiglutide plasma concentration in the period immediately after dialysis was observed, which was matched by increased serum albumin concentrations. These data are consistent with a concentrating effect due to removal of retained body water during the hemodialysis process rather than a direct effect of hemodialysis on the clearance of albiglutide, as this large molecule would not be expected to be cleared during hemodialysis. Similar observations have been noted in the literature and have been attributed to alterations in fluid balance as a result of the hemodialysis process. ^{17, 18} The mechanism for the observed increase of 30 to 40% in albiglutide AUC in patients with severe renal impairment is unclear although it could reflect complex changes in protein metabolism that may occur particularly in patients with type 2 diabetes and end stage renal disease. The clinical impact of such changes as evaluated by the benefit-risk assessment across all grades of renal impairment would suggest that albiglutide could be utilized without the need for dosage modification.

When assessing the benefit of albiglutide, the pooled analysis showed a trend to increased glycemic efficacy as GFR decreased from normal to severe renal impairment. This observation might in part be due to modest increases in albiglutide concentrations and/or may be a function of the multiple metabolic changes that occur as renal function deteriorates. It's reassuring that the effectiveness of albiglutide was not reduced by the duration of diabetes, which was longest in the severe renal impairment group, where a reduction in B-cell function with a loss of insulin secretary activity might be postulated. Evidence is emerging that responsiveness to GLP-1 is not impacted by deterioration in β-cell function and GLP-1 receptor agonists offer the promise of long-term durable glycemic control with the potential to reduce microvascular complications. ²⁹

The adverse event profile for albiglutide in the Phase 3 program was reported elsewhere and was found to be generally safe and well tolerated. ^{10-13,28} Assessment of safety with albiglutide in the present pooled analysis indicated that there was not an association of renal impairment severity with incidence of any adverse events, serious adverse events or adverse events leading to withdrawal. The increased incidence of anemia, renal impairment, and edema in the severe renal impairment group is likely a reflection of the severity of renal disease and not albiglutide therapy.

In the albiglutide Phase 3 program, gastrointestinal events occurred with a slightly higher incidence than placebo.^{12,13} Moreover, within the dedicated renal trial (Harmony 8), ¹⁰ the incidence of diarrhea was higher in the albiglutide group than in the comparator sitagliptin group, but the incidences of nausea and vomiting events were low and similar in both groups. Within the present pooled analysis, there were no new safety concerns identified for albiglutide than were included in similar previous clinical trials.^{10-13,27,28} Trends towards higher amounts of constipation and diarrhea with worsening renal function was observed but it was not clearly seen for nausea and vomiting.

Exenatide and liraglutide have been associated with a number of reports of acute renal injury or exacerbation of CKD. ¹⁹⁻²⁴ It is thought that this acute renal response is secondary to dehydration and loss of fluids due to vomiting and diarrhea, common adverse events associated with GLP-1R agonists. Based on postmarketing surveillance, the incidence of these renal events has resulted in warnings about the use of exenatide and liraglutide in patients with CKD. ^{25, 26} The experience with albiglutide is limited to clinical trials, but in studies in which exenatide ²⁷ or liraglutide ²⁸ was included as a positive control, the incidence of gastroinstestinal AEs was lower in the albiglutide groups compared to the comparator GLP-1R agonists. The nausea (range 7-16%) and vomiting (range 2-5%) percentages for albiglutide from this pooled analysis were comparable to that observed for albiglutide (10% and 5%, respectively) but much less than liraglutide (29% and 9%) in the head-to-head trial of these two GLP-1R agonists. ²⁸ In fact, the albiglutide treated severe renal impaired group only had one subject reporting one episode of vomiting and this event did not lead to withdrawal.

When considering hypoglycemia, the data from the Phase 3 program indicated that albiglutide had a low hypoglycemic potential ^{12,13} which was consistent with its glucose-dependent insulin secretion mechanism. Within the pooled analysis, there were no marked differences in hypoglycemia and most events occurred on a sulphonylurea background medication, which are well known to have a significant hypoglycemic potential.

### Study Limitations

The main limitation of the data was that the pooled analysis included only 19 patients with severe CKD and no subjects on hemodialysis were treated with multiple doses of albiglutide. Due to the small sample size and the potential for variable drug responses in this population, these results for patients with the most severe CKD cannot be considered definitive.

### Conclusions

In conclusion, the totality of the PK, efficacy and safety data indicate that albiglutide has a favorable benefit:risk in patients with T2DM with any degree of renal impairment, including those who require hemodialysis, and the need for a dose adjustment is not expected. Albiglutide has the potential to treat a broad population of patients with T2DM, including those with varying degrees of renal impairment, and this will help with the unmet medical need to have additional safe and effective therapeutic options for renal impaired populations.

### REFERENCES

1. USRDS 2013 Annual Data Report: Atlas of Chronic Kidney Disease and End-Stage Renal Disease in the United States. Bethesda, Md. 2013.
2. Patel A, MacMahon S, Chalmers J, Neal B, Billot L, Woodward M, et al. Intensive blood glucose control and vascular outcomes in patients with type 2 diabetes. N Engl J Med. 2008; 358(24):2560-72.
3. Herrington W G, Nye H J, Aung T. Metformin use in chronic kidney disease: new evidence to guide dosing. QJM. 2013; 106(11):1059-61.
4. Martinez-Castelao A, Gorriz J L, Sola E, Morillas C, Jover A, Coronel F, et al. About the discrepancies between consensus documents, clinical practice guidelines, and legal regulations in the treatment of type 2 diabetes. Nefrologia. 2012; 32(4):419-26.
5. Scheen A J. Pharmacokinetic considerations for the treatment of diabetes in patients with chronic kidney disease. Expert Opin Drug Metab Toxicol. 2013; 9(5):529-50.
6. St Onge E L, Miller S A. Albiglutide: a new GLP-1 analog for the treatment of type 2 diabetes. Expert Opin Biol Ther. 2010; 10(5):801-6.
7. Setji T, Feinglos M. Albiglutide: clinical overview of a long-acting GLP-1 receptor agonist in the treatment of Type 2 diabetes. Expert Rev Endocrinol Metab. 2013; 8(3):229-38.
8. Bush M A, Matthews J E, De Boever E H, Dobbins R L, Hodge R J, Walker S E, et al. Safety, tolerability, pharmacodynamics and pharmacokinetics of albiglutide, a long-acting glucagon-like peptide-1 mimetic, in healthy subjects. Diabetes, obesity & metabolism. 2009; 11(5):498-505.
9. Matthews J E, Stewart M W, De Boever E H, Dobbins R L, Hodge R J, Walker S E, et al. Pharmacodynamics, pharmacokinetics, safety, and tolerability of albiglutide, a long-acting glucagon-like peptide-1 mimetic, in patients with type 2 diabetes. J Clin Endocrinol Metab. 2008; 93(12):4810-7.
10. Leiter L A, Carr M C, Stewart M, Jones-Leone A, Yang F, Handelsman Y, editors. HARMONY 8: Once weekly glucagon-like peptide 1 receptor agonist albiglutide vs sitagliptin for patinets with type 2 diabetes with renal impairment: week 26 results. American Diabetes Association 73rd Scientific Sessions; 2013; Chicago, Ill.
11. Pratley R, Stewart M, Cirkel D, Ye J, Perry C, Can M C, editors. HARMONY 4: 52-Week efficacy of albiglutide (Albi) vs. insulin glargine (Glar) in patients (pts) with T2DM. American Diabetes Association 73rd Scientific Sessions; 2013; Chicago, Ill.
12. Home P, Stewart M, Yang F, Perry C, Can M C, editors. 52-Week efficacy of albiglutide vs placebo and vs pioglitazone in triple therapy (background metformin and glimepiride) in people with type 2 diabetes: HARMONY 5 Study. American Diabetes Association 73rd Scientific Sessions; 2013; Chicago, Ill.
13. Nauck M, Steward M, Perkins C, Jones-Leone A, Yang F, Perry C, et al., editors. HARMONY 2 Week 52 Results: Albiglutide monotherapy in drug-naive patients with type 2 diabetes mellitus. American Diabetes Association 73rd Scientific Sessions; 2013; Chicago, Ill.
14. Inzucchi S E, Bergenstal R M, Buse J B, Diamant M, Ferrannini E, Nauck M, et al. Management of hyperglycemia in type 2 diabetes: a patient-centered approach: position statement of the American Diabetes Association (ADA) and the European Association for the Study of Diabetes (EASD). Diabetes Care. 2012; 35(6):1364-79.
15. Linnebjerg H, Kothare P A, Park S, Mace K, Reddy S, Mitchell M, et al. Effect of renal impairment on the pharmacokinetics of exenatide. Br J Clin Pharmacol. 2007; 64(3):317-27.
16. Jacobsen L V, Hindsberger C, Robson R, Zdravkovic M. Effect of renal impairment on the pharmacokinetics of the GLP-1 analogue liraglutide. Br J Clin Pharmacol. 2009; 68(6):898-905.
17. Jones C H, Wells L, Stoves J, Farquhar F, Woodrow G. Can a reduction in extracellular fluid volume result in increased serum albumin in peritoneal dialysis patients? Am J Kidney Dis. 2002; 39(4):872-5.
18. Chen Y C, Chen H H, Yeh J C. Postdialysis serum albumin is more rational than predialysis value. Nephron. 1999; 82(3):284-5.
19. Ferrer-Garcia J C, Martinez-Chanza N, Tolosa-Torrens M, Sanchez-Juan C. Exenatide and renal failure. Diabet Med. 2010; 27(6):728-9.
20. Johansen O E, Whitfield R. Exenatide may aggravate moderate diabetic renal impairment: a case report. Br J Clin Pharmacol. 2008; 66(4):568-9.
21. Lopez-Ruiz A, del Peso-Gilsanz C, Meoro-Aviles A, Soriano-Palao J, Andreu A, Cabezuelo J, et al. Acute renal failure when exenatide is co-administered with diuretics and angiotensin II blockers. Pharm World Sci. 2010; 32(5):559-61.
22. Weise W J, Sivanandy M S, Block C A, Comi R J. Exenatide-associated ischemic renal failure. Diabetes Care. 2009; 32(2):e22-3.
23. Kaakeh Y, Kanjee S, Boone K, Sutton J. Liraglutide-induced acute kidney injury. Pharmacotherapy. 2012; 32(1):e7-11.
24. Narayana S K, Talab S K, Elrishi M. Liraglutide-induced acute kidney injury. Practical Diabetes. 2012; 29:380-2.
25. Victoza® (liraglutide (rDNA origin) injection. Prescribing Information. Plainsboro, N.J., USA: Novo Nordisk A/S 2013.
26. BYETTA® (exenatide) Injection Prescribing Information. Princeton, N.J.: Briston-Myers Squibb Company 2013.
27. Rosenstock J, Reusch J, Bush M, Yang F, Stewart M, Albiglutide Study G. Potential of albiglutide, a long-acting GLP-1 receptor agonist, in type 2 diabetes: a randomized controlled trial exploring weekly, biweekly, and monthly dosing. Diabetes Care. 2009; 32(10):1880-6.
28. Pratley R E, Barnett A, Feinglos M M, Harman-Boehm I, Nauck M, Ovalle F, et al. Efficacy and safety of once-weekly (QW) albiglutide vs. once-daily (QD) liraglutide in type 2 diabetes (T2D) inadequately controlled on oral agents: Harmony 7 trial. Diabetes. 2012; 61 (Suppl 1):A240-A1.
29 Garber A J, Incretin Effects on b-Cell Function, Replication, and Mass. DIABETES CARE, VOLUME 34, SUPPLEMENT 2, MAY 2011
30. Fast D M, Kelley M, et al. Workshop Report and Follow-Up—AAPS Workshop on Current Topics in GLP Bioanalysis: Assay Reproducibility for Incurred Sample—Implications of Crystal City Recommendations. The AAPS Journal, 2009 June, 11(2): 238-241.

1. A method of treating type 2 diabetes mellitus in a human in need thereof comprising administering a pharmaceutical composition comprising a GLP-1 agonist to said human wherein said human has renal impairment.

2. The method of claim 1, wherein said GLP-1 agonist comprises a polypeptide having at least 95% sequence identity to the amino acid sequence set forth SEQ ID NO:1 over the entire sequence of SEQ ID NO:1.

3. The method of claim 1, wherein said GLP-1 agonist comprises a polypeptide having at least 99% sequence identity to the amino acid sequence set forth SEQ ID NO:1 over the entire sequence of SEQ ID NO:1.

4. The method of claim 1, wherein said GLP-1 agonist consists of a polypeptide having the amino acid sequence set forth in SEQ ID NO:1.

5. The method of claim 1, wherein said GLP-1 agonist consists of a polypeptide having the amino acid sequence set forth in SEQ ID NO:1 which is truncated at the N-terminus and/or the C-terminus by 1, 2, 3, 4, or 5 amino acids.

6. The method of claim 1, wherein said pharmaceutical composition is administered subcutaneously.

7. The method of claim 1 wherein said pharmaceutical composition comprises albiglutide, 2.8% mannitol, 4.2% trehalose dihydrate, 0.01% polysorbate 80, and phosphate buffer at pH 7.2.

8. The method of claim 1, wherein said pharmaceutical composition is administered as a subcutaneous injection selected from the group of at least one 0.32 mL injection, at least one 0.65 mL injection, and at least one 1.0 mL injection.

9. The method of claim 1, wherein said pharmaceutical composition reduces HbA1c in said human.

10. The method of claim 1, wherein said pharmaceutical composition reduces serum glucose in said human.

11. The method of claim 1, wherein the serum half life of said at least one polypeptide having GLP-1 activity is about 5 days.

12. The method of claim 1, wherein said human has received metformin previous to receiving a pharmaceutical composition of the invention.

13. The method of claim 1, wherein said pharmaceutical composition is administered as monothereapy.

14. The method of claim 1, wherein said pharmaceutical composition is co-administered with at least one second hypoglycemic agent.

15. The method of claim 14 wherein said at least one second hypoglycemic agent is selected from: a GLP-1 agonist, incretin hormone, incretin mimetic, agent to increase insulin secretion, sulfonylurea, meglitinide, acetohexamide, chlorpropamide, tolazamide, glipizide, gliclazide, glibenclamide (glyburide), gliquidone, glimepiride, agent to inhibit GLP-1 break down, DPP-IV inhibitor, agent to increase glucose utilization, glitazone, thiazolidinedione, rosiglitazone, pioglitazone, pPAR agonist, agent to reduce hepatic glucose production, metformin, agent to delay glucose absorption, α-glucosidase inhibitor, insulin glargine and/or insulin.

16. The method of claim 1, wherein said pharmaceutical composition comprising a GLP-1 agonist is administered to said human at an initial dose of 30 mg of said GLP-1 agonist and subsequently titrated up to 50 mg of said GLP-1 agonist.

17. A method of treating type 2 diabetes mellitus in a human wherein said human has renal impairment comprising administering to said human a subcutaneous injection of a pharmaceutical composition comprising albiglutide.

18. The method of claim 17, wherein said pharmaceutical composition comprising a albiglutide is administered to said human at an initial dose of 30 mg of albiglutide per week.

19. The method of claim 17, wherein said pharmaceutical composition comprising a albiglutide is administered to said human at an initial dose of 30 mg of albiglutide per week and subsequently titrated up to 50 mg of albiglutide per week.

20. A method of providing glycemic control in a human with type 2 diabetes mellitus and renal impairment, comprising administering to said human a pharmaceutical composition comprising between and including about 30 mg of albiglutide to about 50 mg of albiglutide per week.
